(19) United States
(12) Patent Application Publication
(10) Pub. No.: US 2013/0197535 A1
(43) Pub. Date: Aug. 1, 2013

(54) OVERCOAT TUBE

(71) Applicant: Yuta OKADA, Tokyo (JP)

(72) Inventor: Yuta OKADA, Tokyo (JP)

(73) Assignee: OLYMPUS CORPORATION, Tokyo (JP)

(21) Appl. No.: 13/590,371

(22) Filed: Aug. 21, 2012

Related U.S. Application Data
(63) Continuation of application No. PCT/JP2010/069503, filed on Nov. 2, 2010.

(30) Foreign Application Priority Data
Feb. 25, 2010 (JP) 2010-040005

(51) Int. Cl. A61B 17/00

(52) U.S. Cl. CPC A61B 17/00
USPC 606/108

(57) ABSTRACT

A cannular tube is configured to insert a distal end side thereof into a body cavity to secure an insertion passage through which a medical forceps is introduced from a proximal end side thereof into the body cavity. The overcoat tube includes a distal end-side tube member forming the insertion passage at the distal end side, a proximal end-side tube member forming the insertion passage at the proximal end side, and an intermediate coupler that couples the distal end-side tube member to the proximal end-side tube member so that the distal end-side tube member is capable of being tilted in two axial directions centered on a pivotal center with respect to the proximal end-side tube member and connects the insertion passages of the distal and proximal end-side tube members.

This application is a continuation application based on a PCT patent Application No. PCT/JP2010/069503, filed Nov. 2, 2010, whose priority is claimed on Japanese Patent Application No. 2010-040005, filed Feb. 25, 2010. The contents of both the PCT Application and the Japanese Application are incorporated herein by reference.

### BACKGROUND OF THE INVENTION

1. Field of the Invention

The present invention relates to an overcoat tube, and more particularly, to an overcoat tube through which surgical instruments such as a medical forceps, a treatment tool, and an endoscope are inserted into a body cavity.

2. Description of Related Art

For a surgical operation in the related art, an overcoat tube (trocar) has been used to insert surgical instruments such as a medical forceps, a treatment tool, and an endoscope into a body cavity.

Such an overcoat tube is disclosed in Japanese Unexamined Patent Application, First Publication No. H02-239832, which includes an insertion part having an insertion passage for guiding an endoscope, a treatment tool or the like into a body cavity to use at least part of the insertion passage as a passage supplying air or water into the body cavity. Here, the insertion passage has an inner wall formed in a tapered shape in which an inner diameter thereof is gradually increased from a distal end side thereof toward a proximal end side thereof.

Further, a trocar (overcoat tube) with an angle mechanism is disclosed in Japanese Unexamined Patent Application, First Publication No. H10-262983, which includes an insertion part configured to consecutively connect angle parts capable of being curved in a predetermined direction by pivotably fitting angle rings on a distal end of a hard pipe in sequence, a manipulation part provided on a proximal end of the insertion part, a pivotal member provided on the manipulation part, and a winding wheel connected to the pivotal member. The winding wheel is connected with at least a pair of manipulation wires disposed inside the angle part to have positions of about 180 degrees with respect to each other. A distal end of each manipulation wire is connected to a distal end of the insertion part or in the vicinity of the distal end of the insertion part. A tube through which an insertion member is inserted is further mounted in the insertion part.

In the overcoat tube disclosed in Japanese Unexamined Patent Application, First Publication No. H02-239832, as shown in FIG. 18A, a distal end-side insertion part 100B inserted into a body cavity 103 is in alignment with a proximal end 100A disposed outside the body cavity 103. A medical forceps 101 is inserted into the overcoat tube 100, and a distal end 101a thereof moves into or out of a distal end opening 100a of the insertion part 100B toward a treatment target 104a of an internal organ 104.

Here, when a distance between a body surface 102 and the internal organ 104 at which the treatment target 104a is located is short, a distance between the distal end opening 100a and the treatment target 104a becomes short. In this case, as shown in FIG. 18B, inserting the insertion part 100B in the vicinity of the treatment target 104a and causing the medical forceps 101 to extend from the distal end opening 100a along the surface of the internal organ 104 may be considered.

The trocar (overcoat tube) disclosed in Japanese Unexamined Patent Application, First Publication No. H10-262983 is configured so that the distal end is capable of being curved. For this reason, when the treatment target is located adjacent to the body surface, the angle part is curved by the manipulation wires. Thereby, the distal end of the trocar can be separated from the internal organ, and thus a surgical space can be secured.

The angle part has a multiple joint bending mechanism in which a plurality of angle rings is connected. For this reason, when the angle rings are connected to move the distal end opening in two axial directions and so that pivoting directions of the angle rings vary in an alternating fashion, and when the distal end opening moves in an intermediate direction between the two axial directions, the manipulations of each direction may cause interference.

### SUMMARY OF THE INVENTION

According to a first aspect of the present invention, a cannular tube is configured to insert a distal end side thereof into a body cavity to secure an insertion passage through which a medical instrument is introduced from a proximal end side thereof into the body cavity, and includes: a distal end-side tube member forming the insertion passage at the distal end side; a proximal end-side tube member forming the insertion passage at the proximal end side; and an intermediate coupler that couples the distal end-side tube member to the proximal end-side tube member so that the distal end-side tube member is capable of being tilted in two axial directions centered on one point with respect to the proximal end-side tube member and connects the insertion passages of the distal and proximal end-side tube members.

According to a second aspect of the present invention, the intermediate coupler includes: a support provided at a distal end side of the proximal end-side tube member; a driven part provided at a proximal end side of the distal end-side tube member; and a joint structure that couples the driven part to the support so that the driven part is capable of being tilted in two axial directions centered on one point with respect to the support.

According to a third aspect of the present invention, the joint structure may include a ball joint in which a through-hole is formed.

According to a fourth aspect of the present invention, the joint structure may include a universal joint in which a through-hole is formed.

According to a fifth aspect of the present invention, the joint structure may include a gimbal joint in which a through-hole is formed.

According to a sixth aspect of the present invention, in any one of the second to fifth aspect of the present invention, the intermediate coupler includes a drive mechanism that drives a driving target configured of a movable member or the driven part in the joint structure to thereby tilt the driven part with respect to the support. The drive mechanism is connected with a driving force transfer unit that is remotely manipulated from an outside of the proximal end-side tube member to transfer a driving force.

According to a seventh aspect of the present invention, the drive mechanism includes a pulley that is fixed to the driving target and is pivotably supported on an axis perpendicular to a tilting central axis, and the driving force transfer unit includes a wire wound on the pulley.

According to an eighth aspect of the present invention, the drive mechanism includes a pinion gear that is fixed to the driving target and is pivotably supported on a tilting central axis, and the driving force transfer unit includes a rack engaged with the pinion gear, and a rod-like member moving the rack forward or backward in a given direction.

According to a ninth aspect of the present invention, the drive mechanism includes a link mechanism that is fixed to the driving target at one end thereof and is coupled to the driving force transfer unit at the other end thereof, and the driving force transfer unit includes a rod-like member that moves a link member forward or backward at the other end side of the link mechanism in a given direction.

According to a tenth aspect of the present invention, in any one of the second to fifth embodiment of the present invention, the intermediate coupler includes a movement restriction member and a drive mechanism. The movement restriction member is provided on the proximal end-side tube member and restricts positions of lateral portions of the driven part at positions spaced apart from a tilting center of the joint structure. The drive mechanism drives the movement restriction member to thereby tilt the driven part with respect to the support. The drive mechanism is connected with a driving force transfer unit that is remotely manipulated from an outside of the proximal end-side tube member to transfer a driving force.

According to an eleventh aspect of the present invention, the movement restriction member is coupled to the proximal end-side tube member so that the movement restriction member is capable of pivoting with respect to the proximal end-side tube member and includes a slit in which the lateral portions of the driven part are sandwiched in a circumferential direction of a tilting circle.

According to a twelfth aspect of the present invention, the movement restriction member includes a lateral-portion pressing part that comes into contact with a lateral surface of the driven part and is provided to be able to move forward or backward in an axial direction of the proximal end-side tube member; and a movement guide part that changes a position of the lateral-portion pressing part in a direction perpendicular to the axial direction based on a position of the axial direction of the lateral-portion pressing part, and the driving force transfer unit includes a rod-like member that moves the lateral-portion pressing part forward or backward in the axial direction.

According to a thirteenth aspect of the present invention, the drive mechanism includes two drive systems that tilt the driven part with respect to the support individually in the two axial directions, and the driving force transfer unit includes a dual transfer system that transfers the driving force independently in the two axial directions.

According to a fourteenth aspect of the present invention, a driving force supply unit supplying the driving force to the driving force transfer unit is installed outside of the proximal end-side tube member.

According to a fifteenth aspect of the present invention, the intermediate coupler includes: a flexible intermediate tube member that causes the insertion passages of the distal and proximal end-side tube members to be communicated with each other; a first holding member that is coupled to the proximal end-side tube member so that the first holding member is capable of tilting around a first pivotal axis perpendicular to a central axis of the proximal end-side tube member with respect to the proximal end-side tube member, and that holds lateral portions of a proximal end of the distal end-side tube member to be capable of being tilted around the first pivotal axis; and a second holding member that is coupled to the proximal end-side tube member so that the second holding member is capable of tilting around a second pivotal axis perpendicular to the central axis of the proximal end-side tube member and the first pivotal axis at one point with respect to the proximal end-side tube member, and that holds the lateral portions of the proximal end of the distal end-side tube member to be capable of being tilted around the second pivotal axis.

According to a sixteenth aspect of the present invention, a drive mechanism is provided that drives the first and second holding members each to pivot with respect to the proximal end-side tube member, and the drive mechanism is connected with a driving force transfer unit that is remotely manipulated from an outside of the proximal end-side tube member to transfer a driving force.

### BRIEF DESCRIPTION OF THE DRAWINGS

FIG. 1 is a schematic perspective view showing a configuration and a use state of an overcoat tube according to a first embodiment of the present invention.

FIG. 2A is a schematic perspective view showing a configuration of the overcoat tube according to the first embodiment of the present invention.

FIG. 2B is a cross-sectional view taken along line A-A of FIG. 2A.

FIG. 3A is a schematic perspective view showing a configuration of an overcoat tube according to a first modified example of the first embodiment of the present invention.

FIG. 3B is a cross-sectional view taken along line B-B of FIG. 3A.

FIG. 4A is a schematic perspective view showing a configuration of an overcoat tube according to a second modified example of the first embodiment of the present invention.

FIG. 4B is a cross-sectional view taken along line C-C of FIG. 4A.

FIG. 5 is a schematic perspective view showing a configuration and a use state of an overcoat tube according to a second embodiment of the present invention.

FIG. 6 is a schematic exploded perspective view showing a configuration of an intermediate coupler in the overcoat tube according to the second embodiment of the present invention.

FIG. 7 is a schematic explanatory view showing an operation of the overcoat tube according to the second embodiment of the present invention.

FIG. 8A is a schematic partial cross-sectional view showing a configuration of an intermediate coupler according to a modified example of the second embodiment of the present invention (third modified example).

FIG. 8B is a schematic partial cross-sectional view showing a tilting state of the intermediate coupler according to the modified example of the second embodiment of the present invention (third modified example).

FIG. 9 is a schematic plan view showing a configuration of a driving part according to the modified example of the second embodiment of the present invention (third modified example).

FIG. 10 is a schematic perspective view showing a configuration of a main part in an overcoat tube according to a third embodiment of the present invention.

FIG. 11A is a partial cross-sectional view when viewed from arrow E of FIG. 10.

FIG. 11B is a partial cross-sectional view when viewed from arrow F of FIG. 10.

FIG. 11C is a cross-sectional view taken along line H-H of FIG. 11B.

FIG. 12 is a cross-sectional view taken along line G-G of FIG. 10.

FIG. 13A is a schematic cross-sectional view showing a configuration of a main part in an intermediate coupler according to a modified example of the third embodiment of the present invention (fourth modified example).

FIG. 13B is a schematic cross-sectional view showing a tilting state of the intermediate coupler according to the modified example of the third embodiment of the present invention (fourth modified example).

FIG. 14 is a schematic perspective view showing a configuration of a main part in an overcoat tube according to a fourth embodiment of the present invention.

FIG. 15A is a schematic perspective view showing a configuration of an intermediate tube member in the overcoat tube according to the fourth embodiment of the present invention.

FIG. 15B is a cross-sectional view taken along line K-K of FIG. 15A.

FIG. 16 is a cross-sectional view taken along line J-J of FIG. 14.

FIG. 17 is a schematic plan view showing a configuration of a driving part according to the fourth embodiment of the present invention.

FIG. 18A is a schematic perspective view for explaining an overcoat tube in the related art.

FIG. 18B is a schematic perspective view for explaining the overcoat tube in the related art.

### DETAILED DESCRIPTION OF THE INVENTION

Hereinafter, embodiments of the present invention will be described with reference to the attached drawings. In all the drawings, even when the embodiments are different from each other, the same or equivalent members are assigned the same symbols, and so common description will be omitted.

### First Embodiment

An overcoat tube according to a first embodiment of the present invention will be described.

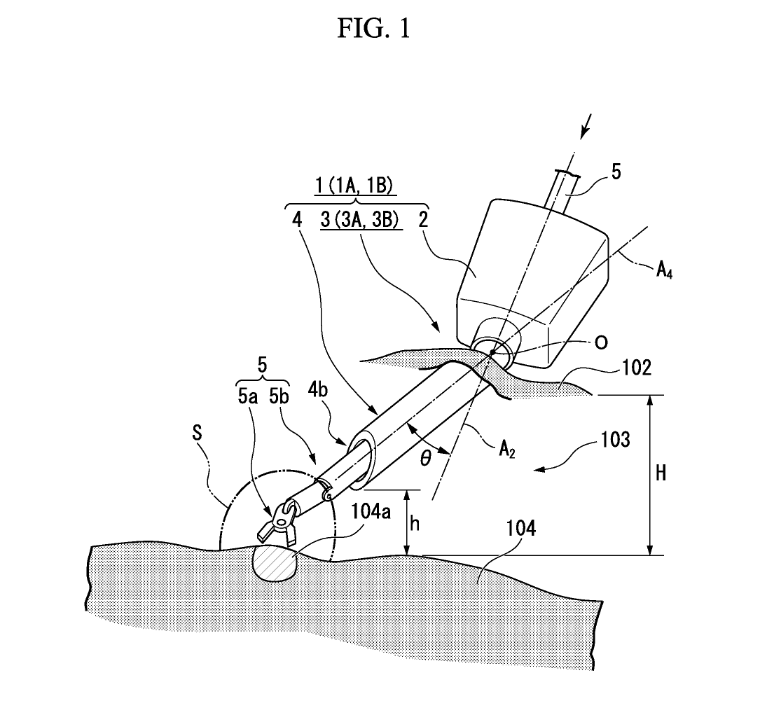

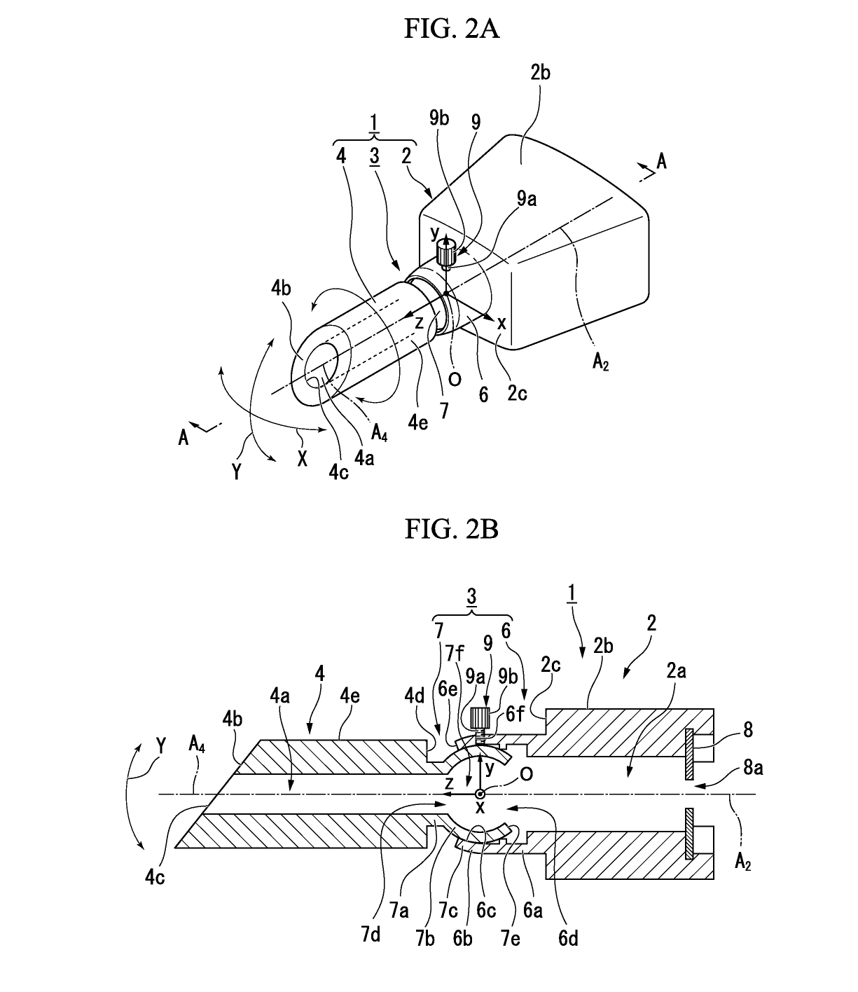

FIG. 1 is a schematic perspective view showing a configuration and a use state of an overcoat tube according to a first embodiment of the present invention. FIG. 2A is a schematic perspective view showing a configuration of the overcoat tube according to the first embodiment of the present invention. FIG. 2B is a cross-sectional view taken along line A-A of FIG. 2A.

Further, each figure is a schematic view, and the size and shape of each member are arbitrarily changed for an easier view (which also applies to the following figures).

As shown in FIGS. 1, 2A, and 2B, the overcoat tube 1 of the present embodiment is configured to insert a distal end side thereof into a body cavity 103 to secure an insertion passage through which a medical instrument is introduced from a proximal end side thereof into the body cavity 103. The overcoat tube 1 includes a distal end-side tube member 4, an intermediate coupler 3, and a proximal end-side tube member 2 from the distal end side thereof toward the proximal end side thereof.

Examples of the medical instrument may include surgical instruments such as a medical forceps, a treatment tool, and an endoscope. In the following description, as an example of this medical instrument, the medical forceps 5 is used.

As shown in FIG. 1, the medical forceps 5 is a rod-like member that is bendably provided in an elongated shape as a whole, and are provided with a forceps distal end 5a at a distal end thereof which pinches or presses a treatment target 104a. The forceps distal end 5a is coupled with a plurality of articular parts 5b, and a bending angle of each articular part 5b is remotely manipulated by a manipulator (not shown) installed outside. Thereby, it is possible to change a position and orientation of the forceps distal end 5a that is introduced from the distal end side of the overcoat tube 1 into the body cavity 103.

The distal end-side tube member 4 is approximately a circular tubular member provided therethrough with a circular hole-shaped insertion passage 4a into which the medical forceps 5 is inserted. The distal end-side tube member 4 is used to at least insert the distal end side thereof into the body cavity 103. An inner diameter of the insertion passage 4a is set to at least a diameter that is greater than an outer diameter of the medical forceps 5 inserted into the overcoat tube 1.

The distal end of the distal end-side tube member 4 includes a slanted face 4b that is a plane intersecting slantly with a distal end-side tube member central axis A₄ that is a common central axis of the distal end-side tube member 4 and the insertion passage 4a, and has a tapered needle shape when viewed from the side. Thereby, the distal end of the insertion passage 4a is provided with an elliptical distal end opening 4c. For this reason, the distal end opening 4c is opened toward an axial distal end side thereof as well as toward a direction lateral to the axial direction.

A proximal end of the distal end-side tube member 4 is provided with a proximal end face 4d that is a plane perpendicular to the distal end-side tube member central axis A₄.

The distal end-side tube member 4 may have a cylindrical tubular shape in which an outer diameter of an outer circumferential surface 4e thereof and an inner diameter of the insertion passage 4a are constant in the axial direction thereof, or may be formed in a conical surface shape in which at least one of the outer diameter of the outer circumferential surface 4e and the inner diameter of the insertion passage 4a is reduced from the proximal end side toward the distal end side thereof.

The intermediate coupler 3 couples the distal end-side tube member 4 to the proximal end-side member 2 so that the distal end-side tube member 4 is capable of being tilted with respect to the proximal end-side tube member 2 in two axial directions that are centered on a pivotal center O, and simultaneously communicates the insertion passages the distal end-side tube member 4 and proximal end-side tube member 2 with each other.

The phrase “to be able to be tilted in two axial directions that are centered on a pivotal center O” refers to being capable of being tilted around the pivotal center O in an arbitrary axial direction passing through the pivotal center O on a plane perpendicular to a tilting central axis (proximal end-side tube member central axis A₂ as will be described below) passing through the pivotal center O.

In the present embodiment, the intermediate coupler 3 is configured of a male coupler 7 installed on the proximal end of the distal end-side tube member 4 and a female coupler 6 installed on the distal end of the proximal end-side tube member 2. Although not shown in FIG. 1, the intermediate coupler 3 is provided with a handled screw 9, as shown in FIGS. 2A and 2B, in order to fix and release a relative position between the male coupler 7 and the female coupler 6.

The male coupler 7 is provided with a tubular part 7a that is connected to the proximal end face 4d of the distal end-side tube member 4, and a spherical shell-shaped male joint part 7b that is connected to a proximal end side of the tubular part 7a.

In the tubular part 7a a through-hole 7d that has the same inner diameter as the insertion passage 4a of the proximal end side of the distal end-side tube member 4 is provided.

In the present embodiment, an external form of the tubular part 7a is a cylindrical shape, but it may be a tetragonal prism shape.

An outer circumferential surface of the male joint part 7b is provided with a convex engaging surface 7c that is a partial spherical surface whose outer diameter is greater than that of the tubular part 7a.

In the male joint part 7b, a spherical cavity part 7f that communicates with the through-hole 7d of the tubular part 7a at a distal end side thereof is provided.

The male joint part 7b is provided with a circular hole-shaped opening 7e at a proximal end side thereof which is centered on the distal end-side tube member central axis A₄ and has an inner diameter that is greater than that of the through-hole 7d.

The female coupler 6 includes a tubular part 6a connected to the distal end of the proximal end-side tube member 2, and a partial spherical shell-shaped female joint part 6b connected to a distal end side of the tubular part 6a.

In the tubular part 6a, a through-hole 6d, an inner diameter of which is greater than that of the insertion passage 4a of the distal end-side tube member 4, is provided.

In the present embodiment, an external form of the tubular part 6a is a cylindrical shape, but it may be a tetragonal prism shape.

The female joint part 6b is provided with a concave engaging surface 6c on an inner side thereof which is a partial spherical surface fitted slidably onto the convex engaging surface 7c of the male coupler 7.

The female joint part 6b is provided with a circular hole-shaped opening 6e on a distal end side thereof which is centered on the proximal end-side tube member central axis A₂ of the proximal end-side tube member 2 to be described below and which has a greater inner diameter than an outer diameter of the tubular part 7a of the male coupler 7.

The female joint part 6b is provided with a female threaded part 6f which passes through a lateral surface thereof and which is screwed with the handled screw 9.

The handled screw 9 is provided with a male threaded part 9a screwed with the female threaded part 6f provided on the female joint part 6b, and a handle part 9b performing the screw fastening of the male threaded part 9a. In the present embodiment, the handle part 9b is configured of approximately a cylindrical member having a knurled surface whose diameter is greater than that of the male threaded part 9a. A tip of the male threaded part 9a has a flat point shape that can press the male joint part 7b without damage when screw-fastened and contacted with the male joint part 7b.

As shown in FIGS. 1 and 2A, the proximal end-side tube member 2 is a tubular member, which has approximately a tetragonal pyramidal frustum-shaped external form 2b that is slightly narrowed toward the distal end side thereof, through which a circular hole-shaped insertion passage 2a into which the medical forceps 5 is inserted is provided, and at distal end side of which the female coupler 6 is provided.

The insertion passage 2a is configured to set an inner diameter thereof to be greater than at least an outer diameter of the medical forceps 5 inserted into the overcoat tube 1, and to be coaxial with an insertion hole 6d of the female coupler 6 at the distal end side thereof, as shown in FIG. 2B.

The central axis of the insertion passage 2a is consistent with the proximal end-side tube member central axis A₂ that is the central axis of the external form of the proximal end-side tube member 2. For this reason, the pivotal center O of the female coupler 6 is located on the proximal end-side tube member central axis A₂.

An external form of the distal end side of the proximal end-side tube member 2 has a rectangular shape that is greater than the outer diameter of the female coupler 6. Thereby, a stepped part 2c is formed between an outer circumference of the tubular part 6a of the female coupler 6 and a tube outer circumference of the proximal end-side tube member 2 as a plane that is perpendicular to the proximal end-side tube member central axis A₂.

The overcoat tube 1 of the present embodiment sets a position at which the stepped part 2c comes into close contact with a body surface 102 as an insertion limit. For this reason, the proximal end-side tube member 2 is disposed and used at least outside the body cavity 103.

The insertion passage 2a is provided with an airtight valve 8 at a proximal end side thereof which keeps the inside of the insertion passage 2a in an airtight and liquid-tight manner with the medical forceps 5 inserted thereinto. The airtight valve 8 is formed of a material such as a synthetic rubber having elasticity. The airtight valve 8 has a smaller inner diameter than the outer diameter of the medical forceps 5, and is provided with a hole part 8a that comes into close contact with an external form of the medical forceps 5 when the medical forceps 5 is inserted.

With this configuration, the male joint part 7b of the male coupler 7 is fitted into the female joint part 6b of the female coupler 6, as shown in FIG. 2B. Thereby, the female coupler 6 and the male coupler 7 are slidably engaged by the concave engaging surface 6c and the convex engaging surface 7c, respectively. Further, when the handled screw 9 is fastened, the relative position between the female coupler 6 and the male coupler 7 is fixed. When the handled screw 9 is loosened, the female coupler 6 and the male coupler 7 are kept unfixed.

In this unfixed state, the male coupler 7 freely pivots around the pivotal center O that is a common center of curvature of the concave engaging surface 6c and the convex engaging surface 7c within a range in which the external form of the tubular part 7a is in contact with an inner edge of the opening 6e.

Thereby, the distal end-side tube member 4 connected to the male coupler 7 can be tilted with respect to the proximal end-side tube member central axis A₂ adopting the pivotal center O as a tilting center.

For example, as shown in FIG. 2A, when two axes perpendicular to each other on the plane that passes through the pivotal center O and orthogonally intersects with the proximal end-side tube member central axis A₂ are x and y axes, and when an axis perpendicular to the x and y axes is a z axis, the z axis is consistent with the proximal end-side tube member central axis A₂, and the x and y axes become two tiltable axial directions that are centered on the pivotal center O.

For example, when the distal end-side tube member 4 is tilted around the x axis, the distal end-side tube member 4 is tilted within an yz plane in a direction along the y axis, as indicated by an arrow Y.

Further, when the distal end-side tube member 4 is tilted around the y axis, the distal end-side tube member 4 is tilted within a zx plane in a direction along the x axis, as indicated by an arrow X.

The male coupler 7 and the female coupler 6 are configured so that the engaging surfaces thereof are spherical surfaces, and there are no limitations in the pivoting direction. For this reason, the distal end-side tube member 4 can be tilted within a xy plane in an arbitrary axial direction that passes through the pivotal center O, i.e., can be tilted in the two axial directions.

Further, when the handled screw 9 is fastened, the relative position between the female coupler 6 and the male coupler 7 is fixed, so that a tilted positional relationship can be maintained.

The cavity part 7f and the through-hole 7d inside the male coupler 7 communicate with the insertion passage 4a. The opening 7e is open toward the through-hole 6d of the female coupler 6 regardless of the pivoted state of the male coupler 7. For this reason, the insertion passage 4a is communicated with the insertion passage 2a via the through-hole 7d, the cavity part 7f, and the through-hole 6d. Thereby, in the overcoat tube 1, an insertion passage that runs through in an axial direction is provided.

As materials for the distal end-side tube member 4, the intermediate coupler 3, and the distal end-side tube member 4, an arbitrary synthetic resin or a combination of a synthetic resin and a metal may be employed.

In this manner, the overcoat tube 1 of the present embodiment includes the distal end-side tube member 4 having the insertion passage 4a at the distal end side thereof, the proximal end-side tube member 2 having the insertion passage 2a at the proximal end side thereof, and the intermediate coupler 3 that couples the distal end-side tube member 4 to the proximal end-side tube member 2 so that the distal end-side tube member 4 is capable of being tilted with respect to the proximal end-side tube member 2 in the two axial directions that are centered on the pivotal center O and that causes the insertion passages 4a and 2a of the distal and proximal end-side tube members 4 and 2 to be communicated via the through-hole 7d, the cavity part 7f, and the through-hole 6d.

Further, the intermediate coupler 3 includes the tubular part 6a that is a support provided at the distal end side of the proximal end-side tube member 2, and the tubular part 7a that is a driven part provided at the proximal end side of the distal end-side tube member 4, and has a joint structure in which the tubular part 7a is coupled to the tubular part 6a to be capable of being tilted in the two axial directions centered on the pivotal center O.

This joint structure is configured of a ball joint in which a through-hole is formed.

Next, an operation of the overcoat tube 1 of the present embodiment will be described.

The overcoat tube 1 is configured so that the distal end-side tube member 4 and the proximal end-side tube member 2 are coupled to be capable of being tilted in two axial directions centered on one point by the intermediate coupler 3 configured of a ball joint. For this reason, as shown in FIG. 1, the distal end-side tube member central axis A₄ can be tilted with respect to the proximal end-side tube member central axis A₂ of the proximal end-side tube member 2 whose position is fixed outside the body surface 102.

Thereby, even when a distance between the body surface 102 and the internal organ 104 at which the treatment target 104a is located is short, the distal end-side tube member 4 can be inserted in a state in which a distance between the distal end of the distal end-side tube member 4 and the internal organ 104 is sufficiently secured, and a necessary surgical space S can be secured between the distal end of the distal end-side tube member 4 and the treatment target 104a in order to allow the medical forceps 5 to extend above the treatment target 104a and to be freely moved by the articular part 5b.

For example, depending on a size of the necessary surgical space S, a height h from the internal organ 104 to dispose the distal end of the distal end-side tube member 4 is determined, and depending on a height H from the body surface 102 to the internal organ 104, an angle θ to tilt the distal end-side tube member 4 is determined. Accordingly, the distal end-side tube member 4 is tilted in advance by the angle θ, and thus an inserting position is similarly determined. The overcoat tube 1 may be inserted from the inserting position.

Alternatively, the intermediate coupler 3 may be kept freely pivoted, and the medical forceps 5, which is inserted up to the insertion passage 4a of the distal end-side tube member 4, may be curved by remote manipulation from the outside. Thereby, the tilting direction and amount of the tilting of the distal end-side tube member 4 may be controlled. In this case, the tilting angle may be changed while performing the inserting movement.

When an endoscope is inserted in place of the medical forceps 5, the endoscope is curved while an image in front of the distal end opening 4c is being observed by the endoscope. Thereby, the tilting angle can be adjusted.

Further, in any case, the handled screw 9 is fastened with proper timing, so that the tilting angle can be maintained at that time. Thereby, the position of the distal end opening 4c of the overcoat tube 1 can be stabilized to perform the treatment.

The intermediate coupler 3 of the overcoat tube 1 has the joint structure, so that the intermediate coupler 3 can be tilted around the pivotal center O at one joint. For this reason, for example, in comparison with a case in which the same tilting angle is obtained by an multiple joint bending mechanism in which a plurality of joint rings are pivotably coupled, an axial length can be shortened, and a length of the overcoat tube 1 can be shortened.

In this manner, the overcoat tube 1 is provided with the intermediate coupler 3 that couples the distal end-side tube member 4 with the proximal end-side tube member 2 so that the distal end-side tube member 4 is capable of being tilted with respect to the proximal end-side tube member 2 in the two axial directions centered on the pivotal center O, and that communicates the insertion passages 4a and 2a of the distal and proximal end-side tube members 4 and 2 with each other. For this reason, the surgical space S can be widely secured even for the treatment target 104a located in shallow position from the body surface 102 by simple manipulation.

### First Modified Example

Next, a first modified example of the present embodiment will be described.

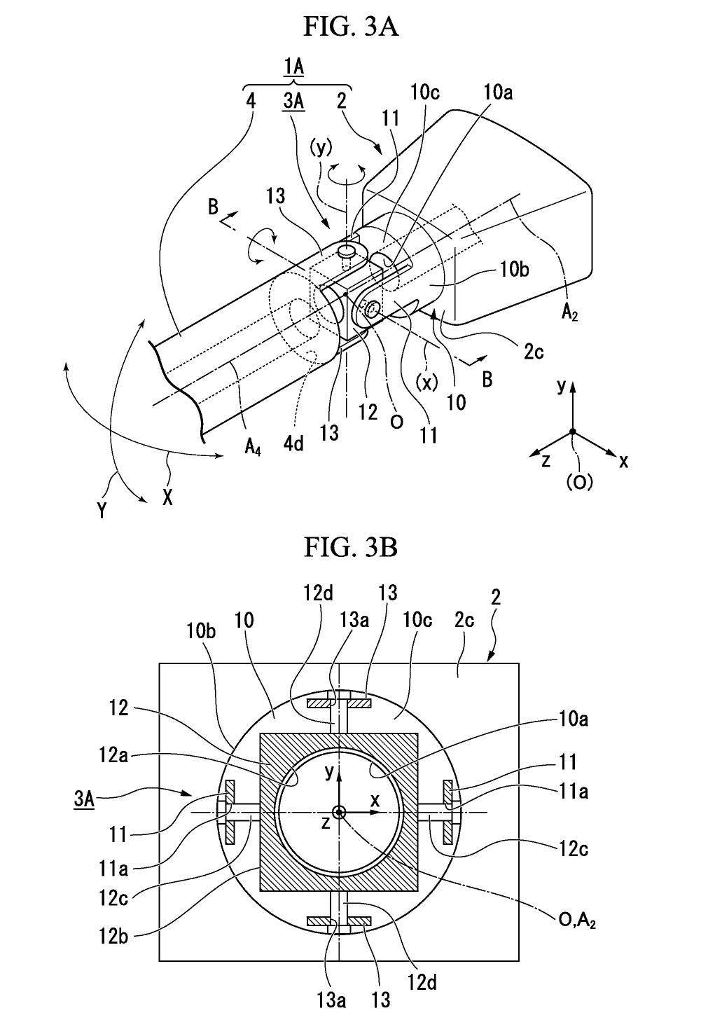

FIG. 3A is a schematic perspective view showing a configuration of an overcoat tube according to a first modified example of the first embodiment of the present invention. FIG. 3B is a cross-sectional view taken along line B-B of FIG. 3A. In FIG. 3A, for a clearer view, positions of xyz axes to be depicted adopting a pivotal center O as an origin are depicted with slight displacement.

An overcoat tube 1A of the present modified example is provided with an intermediate coupler 3A in place of the intermediate coupler 3 of the overcoat tube 1 of the first embodiment. The following description will focus on differences between the present modified example and the first embodiment.

As shown in FIGS. 3A and 3B, similar to the intermediate coupler 3, the intermediate coupler 3A couples a distal end-side tube member 4 to a proximal end-side tube member 2 so that the distal end-side tube member 4 is capable of being tilted with respect to a proximal end-side tube member 2 in two axial directions centered on the pivotal center O, and simultaneously communicates insertion passages 4a and 2a of the distal and proximal end-side tube members 4 and 2 with each other.

The intermediate coupler 3A is provided with a pair of wing parts (driven parts) 13 formed on a proximal end face 4d of the distal end-side tube member 4, a tubular support (support) 10 formed on a stepped part 2c of the proximal end-side tube member 2, a pair of wing parts 11 formed on a distal end side of the tubular support 10, and a case member 12 coupling the wing parts 13 and 11.

The pair of wing parts 13 is opposite to each other at an outer edge of the proximal end face 4d in a radial direction of the distal end-side tube member 4, and are tabular members extending along a distal end-side tube member central axis A₄.

A protrusion-directional end of each wing part 13 is provided with a hole 13a which passes therethrough in a thickness-wise direction and which is coaxially formed on an axis (y axis as shown) that passes through the pivotal center O and is perpendicular to the distal end-side tube member central axis A₄.

The tubular support 10 protrudes from the stepped part 2c along a proximal end-side tube member central axis A₂, and a protrusion-directional distal end thereof is provided with a distal end face 10c that consists of a plane perpendicular to the proximal end-side tube member central axis A₂.

Further, in the tubular support 10, a through-hole 10a which communicates with the insertion passage 2a and which has the same axis as the insertion passage 2a is provided.

In the present modified example, to cause the pair of wing parts 13 to be opposite to the pair of wing parts 11 at approximately the same interval, an outer circumferential surface 10b of the tubular support 10 is formed in a cylindrical shape having approximately the same outer diameter as the proximal end side of the distal end-side tube member 4.

However, the outer diameter of the outer circumferential surface 10b may be different from that of the distal end-side tube member 4. Further, the shape of the outer circumferential surface 10b is not limited to the cylindrical shape, and it may be a tetragonal prism shape.

The pair of wing parts 11 is opposite to each other at an outer edge of the distal end face 10c in a radial direction of the tubular support 10, and are tabular members extending along the proximal end-side tube member central axis A₂.

A protrusion-directional end of each wing part 11 is provided with a hole 11a which passes therethrough in a thickness-wise direction and which is coaxially formed on an axis (x axis as shown) that passes through the pivotal center O and is perpendicular to the distal end-side tube member central axis A₄.

The case member 12 is configured so that a through-hole 12a having an inner diameter allowing medical forceps 5 to be inserted thereinto is formed in the center of a rectangular block member having a narrower width than an interval between the opposed wing parts 11 and 13, and so that a pair of pivot shafts 12c and a pair of pivot shafts 12d are erected on four outer circumferential surfaces 12b surrounding the through-hole 12a in directions that are perpendicular to the outer circumferential surfaces 12b.

The pivot shafts 12c and 12d are shaft members whose tips are pivotably attached to the holes 11a and 13a, respectively, and are installed at the centers of the respective outer circumferential surfaces 12b.

For this reason, the pair of pivot shafts 12c are arranged on one axis (x axis as shown) that is perpendicular to a central axis of the through-hole 12a, and the pair of pivot shafts 12d are arranged on an axis (y axis as shown) that is perpendicular to the central axis of the through-hole 12a and the axis on which the pair of pivot shafts 12c are arranged at one point.

Further, the case member 12 is pivotably coupled to the wing parts 13 in a state in which the pivot shafts 12d are inserted from the inside into the holes 13a of the wing parts 13, and are pivotably coupled to the wing parts 11 in a state in which the pivot shafts 12c are inserted from the inside into the holes 11a of the wing parts 11.

With this configuration, a first pivotal part is formed between the distal end-side tube member 4 having the wing parts 13 and the case member 12 to allow the distal end-side tube member 4 to pivot around the shown y axis (first pivotal axis) perpendicular to the distal end-side tube member central axis A₄ with respect to the case member 12.

Further, a second pivotal part is formed between the tubular support 10 having the wing parts 11 and the case member 12 to allow the case member 12 to pivot around the central axis of the through-hole 10a of the tubular support 10, i.e. the shown x axis (second pivotal axis) perpendicular to the proximal end-side tube member central axis A₂, with respect to the tubular support 10.

Thereby, the distal end-side tube member 4 having the wing parts 13 can be tilted in two axial directions with respect to the proximal end-side tube member central axis A₂ adopting the pivotal center O intersecting with the x and y axes as a tilting center (see arrows X and Y of FIG. 3A), as in the first embodiment.

That is, however the distal end-side tube member 4 pivots around the pivot shafts 12c with respect to the case member 12, the case member 12 and the distal end-side tube member 4 coupled to the case member 12 can pivot around the x axis that is the same axis as the pivot shafts 12d.

Furthermore, however the case member 12 pivots around the x axis with respect to the wing parts 11, the distal end-side tube member 4 can pivot around the pivot shafts 12c.

Since the pivot shafts 12c and 12d are perpendicular to each other at the pivotal center O, the pivoting motions of the two axial directions are not influenced by each other. For this reason, the distal end-side tube member 4 can be tilted in directions of the x- and y-axial directions perpendicular to the pivotal center O, and in an arbitrary axial direction passing through the pivotal center O within the xy plane.

The joint structure using this case member 12 becomes a universal joint. The universal joint constitutes one joint that pivots in two axial directions.

The through-hole 12a of the case member 12 is sandwiched between the insertion passages 4a and 2a of the distal and proximal end-side tube members 4 and 2 which are coupled by the case member 12, and thus is located at a position where the center thereof intersects with the distal end-side tube member central axis A₄ and the proximal end-side tube member central axis A₂. For this reason, even when the distal end-side tube member 4 is tilted, the distal end-side tube member central axis A₄ and the proximal end-side tube member central axis A₂ pass through the center of the through-hole 12a. As such, the through-hole 12a becomes a through-hole that communicates the insertion passages 4a and 2a with each other.

In the overcoat tube 1A of the present modified example, the intermediate coupler 3A has the joint structure that provides coupling capable of being tilted around the pivotal center O in the two axial directions, like the intermediate coupler 3. For this reason, the overcoat tube 1A has a function similar to the overcoat tube 1 of the first embodiment.

### Second Modified Example

Next, a second modified example of the present embodiment will be described.

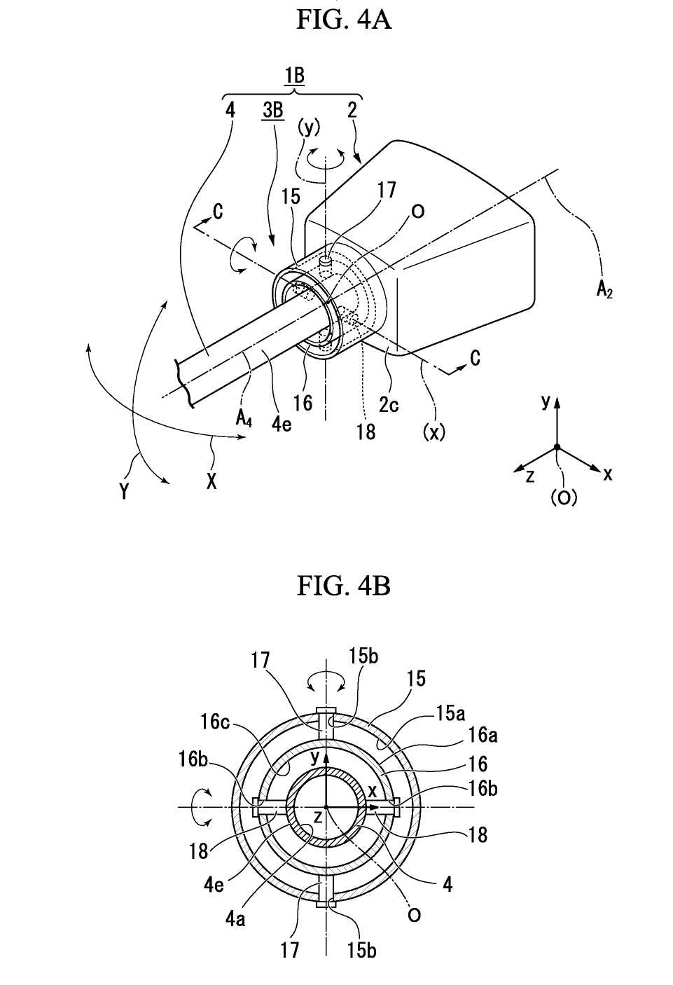

FIG. 4A is a schematic perspective view showing a configuration of an overcoat tube according to a second modified example of the first embodiment of the present invention. FIG. 4B is a cross-sectional view taken along line C-C of FIG. 4A. In FIG. 4A, for a clearer view, positions of xyz axes to be depicted using a pivotal center O as an origin are depicted with slight displacement.

An overcoat tube 1B of the present modified example is provided with an intermediate coupler 3B in place of the intermediate coupler 3 of the overcoat tube 1 of the first embodiment. The following description will focus on differences between the present modified example and the first embodiment.

As shown in FIGS. 4A and 4B, similar to the intermediate coupler 3, the intermediate coupler 3B couples a distal end-side tube member 4 to a proximal end-side tube member 2 so that the distal end-side member 4 is capable of being tilted with respect to a proximal end-side tube member 2 in two axial directions centered on the pivotal center O, and simultaneously communicates insertion passages 4a and 2a of the distal and proximal end-side tube members 4 and 2 with each other.

The intermediate coupler 3B is provided with an outer case 15 installed on a stepped part 2c of the proximal end-side tube member 2, and an intermediate case member 16 disposed on an inner side of the outer case 15 and on a radial outer side of the distal end-side tube member 4.

The outer case 15 of the modified example is an annular case member that protrudes from the stepped part 2c along a proximal end-side tube member central axis A₂.

In the outer case 15, a through-hole 15a which communicates with the insertion passage 2a is provided concentrically with the proximal end-side tube member central axis A₂.

Further, the outer case 15 is provided with two holes 15b in a lateral portion thereof, each of which has the center located on one axis (y axis as shown), which passes through the center of the through-hole 15a and extends in a radial direction. The two holes 15b radially pass through the outer case 15.

The intermediate case member 16 of the present modified example is an annular case member, an axial width of which is narrower than that of the outer case 15 and in which a through-hole 16c is provided.

The intermediate case member 16 is provided with a pair of pivot shafts 17, each of which extends from an outer circumferential surface 16a thereof toward a radial outer side in one axial direction perpendicular to a central axis of the intermediate case member 16.

Tips of the pivot shafts 17 are located at positions spaced apart from the outer circumferential surface 16a by the same distance, and are pivotably coupled to the holes 15b of the outer case 15.

Further, the intermediate case member 16 is provided with two holes 16b, each of which has the center set on an axis (x axis as shown) that are perpendicular to the central axis of the intermediate case member 16 and to an axis on which the pivot shafts 17 are arranged. The two holes 16b radially pass through the intermediate case member 16.

The distal end-side tube member 4 of the present modified example is configured so that an outer diameter of a proximal end-side outer circumferential surface 4e thereof is smaller than an inner diameter of the intermediate case member 16.

The distal end-side tube member 4 is provided with a pair of pivot shafts 18, each of which extends from the outer circumferential surface 4e toward a radial outer side in one axial direction perpendicular to a distal end-side tube member central axis A₄.

Tips of the pivot shafts 18 are located at positions spaced apart from the outer circumferential surface 4e by the same distance, and are pivotably coupled to the holes 16b of the intermediate case member 16.

According to the intermediate coupler 3B as configured in this way, the outer case 15 constitutes a support provided at a distal end side of the proximal end-side tube member 2, and an end of the distal end-side tube member 4 having the pivot shafts 18 constitutes a driven part provided at a proximal end side of the distal end-side tube member 4. The present modified example is illustrative of a case in which the support is the outer case allowed to be disposed outside the driven part, and in which the driven part is an inner case allowed to be disposed inside the support.

The pivot shafts 17 constitute a first pivotal axis that is provided on a plane perpendicular to the central axis of the intermediate case member 16. The holes 15b constitute an outside pivotal part that couples the intermediate case member 16 and the outer case 15 so as to be capable of pivoting around the first pivotal axis.

The pivot shafts 18 constitute a second pivotal axis provided on a plane perpendicular to the central axis of the intermediate case member 16 to be perpendicular to the first pivotal axis. The holes 16b constitute an inside pivotal part that couples the intermediate case member 16 and the proximal end of the distal end-side tube member 4 that is the inner case to be able to pivot around the second pivotal axis.

Thereby, the distal end-side tube member 4 can be tilted in two axial directions with respect to the proximal end-side tube member central axis A₂ adopting the pivotal center O intersecting with the shown x and y axes as a tilting center (see arrows X and Y of FIG. 4A), as in the first embodiment.

That is, however the distal end-side tube member 4 pivots around the pivot shafts 18 with respect to the intermediate case member 16, the intermediate case member 16 and the distal end-side tube member 4 coupled to the intermediate case member 16 can pivot around the y axis that is the same axis as the pivot shafts 17.

Further, however the intermediate case member 16 pivots around the y axis with respect to the outer case 15, the distal end-side tube member 4 can pivot around the pivot shafts 18.

The pivot shafts 17 and 18 are perpendicular to the pivotal center O. Accordingly, since the pivoting motions of the two axial directions are not influenced by each other, the distal end-side tube member 4 can be tilted in directions of the x- and y-axial directions perpendicular to the pivotal center O, and in an arbitrary axial direction passing through the pivotal center O within the xy plane.

The joint structure using this intermediate case member 16 becomes a gimbal joint which uses a gimbal mechanism and has a through-hole therein. The gimbal joint constitutes one joint that pivots in two axial directions.

Further, in the present modified example, the insertion passage 4a of the proximal end side of the distal end-side tube member 4 is disposed inside the intermediate case member 16 and the outer case 15 at any tilting position. For this reason, the insertion passage 4a communicates with the insertion passage 2a via the through-hole 15a.

According to the overcoat tube 1B of the present modified example, the intermediate coupler 3B has the joint structure that provides coupling to be able to be tilted around the pivotal center O in the two axial directions, like the intermediate coupler 3. For this reason, the overcoat tube 1B has a function similar to the overcoat tube 1 of the first embodiment.

### Second Embodiment

An overcoat tube according to a second embodiment of the present invention will be described.

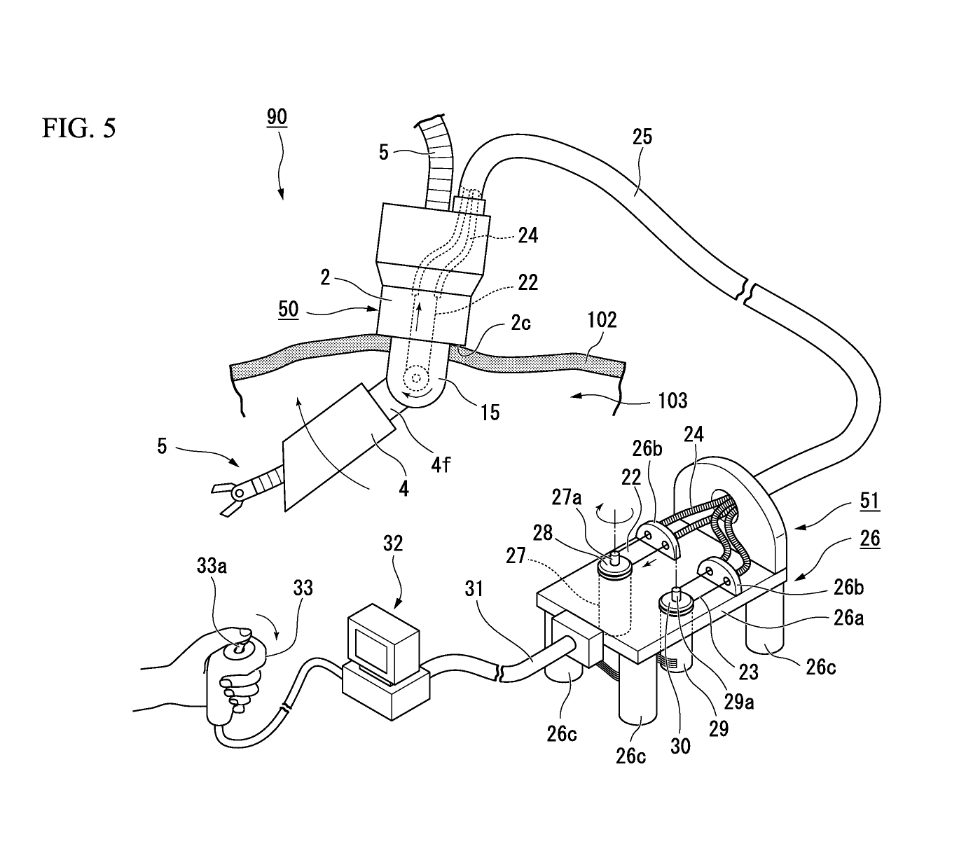

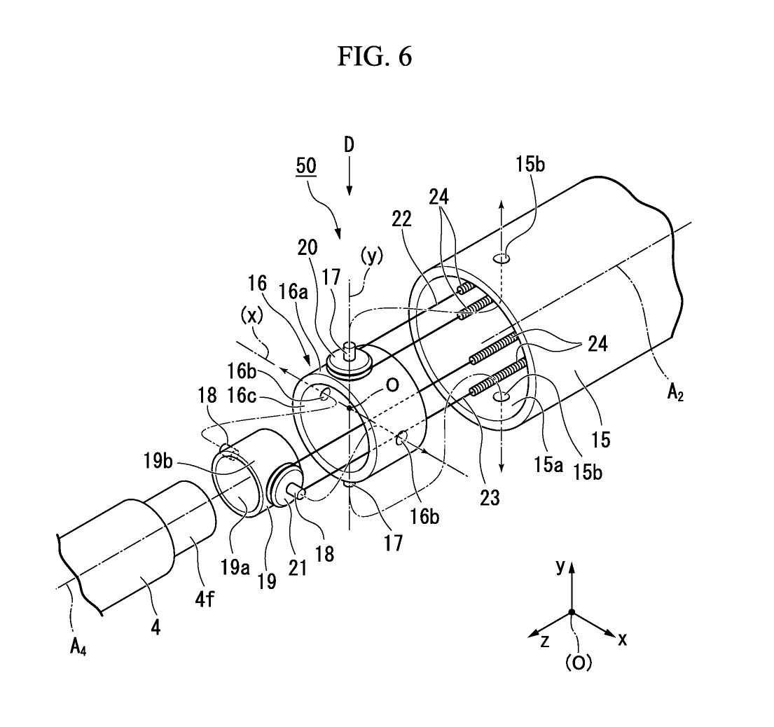

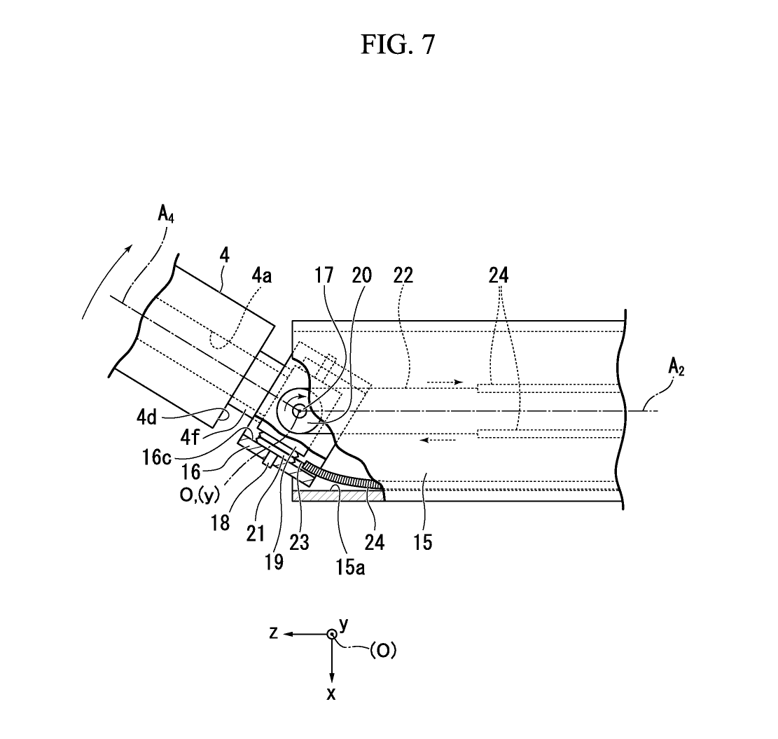

FIG. 5 is a schematic perspective view showing a configuration and a use state of an overcoat tube according to a second embodiment of the present invention. FIG. 6 is a schematic exploded perspective view showing a configuration of an intermediate coupler in the overcoat tube according to the second embodiment of the present invention. In FIG. 6, for a clearer view, positions of xyz axes to be depicted using a pivotal center O as an origin are depicted with slight displacement (hereinafter, also equally applied to FIG. 7).

As shown in FIGS. 5 and 6, the overcoat tube 90 of the present embodiment is configured to insert a distal end side thereof into a body cavity 103 to secure an insertion passage that introduces medical forceps 5 from a proximal end side thereof into the body cavity 103. The overcoat tube 90 includes a distal end-side tube member 4, an intermediate coupler 50, and a proximal end-side tube member 2 from the distal end side thereof toward the proximal end side thereof, and further includes a driving part 51. The following description will focus on differences between the second embodiment and the first embodiment.

Like the intermediate coupler 3B of the overcoat tube 1B of the second modified example of the first embodiment, the intermediate coupler 50 includes an outer case 15 and an intermediate case member 16. The pivot shafts 18 of the overcoat tube 1B of the second modified example of the first embodiment are directly attached to the outer circumferential surface 4e of the proximal end side of the distal end-side tube member 4. In contrast, in the present embodiment, the distal end-side tube member 4 is provided with an annular connecting pipe part 4f at a proximal end side thereof, and an annular inner case 19 through which a connecting hole 19a passes is fixedly fitted onto the connecting pipe part 4f. An outer circumferential surface 19b of the inner case 19 is provided with a pair of pivot shafts 18.

The outer circumferential surface 19b of the inner case 19 is configured of a cylindrical surface having the same outer diameter as the outer circumferential surface 4e of the distal end-side tube member 4 of the second modified example of the first embodiment.

The pivot shafts 18 installed on the inner case 19 are located at positions spaced apart from the outer circumferential surface 19b by the same distance, and are pivotably coupled to holes 16b of the intermediate case member 16. Further, the intermediate case member 16 is pivotably coupled to the outer case 15 via pivot shafts 17, like the intermediate coupler 3B.

However, unlike the intermediate coupler 3B, the intermediate coupler 50 is configured so that pulleys 20 and 21, which are disposed between the outer circumferential surface 16a and an inner circumferential surface of the through-hole 15a and between the outer circumferential surface 19b and an inner circumferential surface of the through-hole 16c, are fixed to one of the pivot shafts 17 and one of the pivot shafts 18, respectively.

For this reason, when the pulleys 20 and 21 turn around the respective pivot shafts 17 and 18, the intermediate case member 16 and the inner case 19 to which the pivot shafts 17 and 18 are fixed pivot by receiving rotational driving forces of the pivot shafts 17 and 18.

According to the intermediate coupler 50 as configured in this way, the inner case 19 having the pivot shafts 18 constitutes a driven part provided at a proximal end side of the distal end-side tube member 4. For this reason, similar to the intermediate coupler 3B, a first pivotal axis, an outside pivotal part, a second pivotal axis, and an inside pivotal part are formed, and a joint structure made up of a gimbal joint in which a through-hole is provided is formed.

Accordingly, although not shown specifically, the distal end-side tube member 4 can be tilted in two axial directions with respect to a proximal end-side tube member central axis A₂ using a pivotal center O similar to that of the intermediate coupler 3B as a tilting center.

Furthermore, the pulleys 20 and 21 of the intermediate coupler 50 of the present embodiment constitute a drive mechanism that tilts the driven part with respect to a support.

As shown in FIG. 6, to pivot the pulleys 20 and 21, the pulleys 20 and 21 are wound with wires 22 and 23 having the same outer diameter in an endless ring shape, respectively.

As a material for the wires 22 and 23, a metal, a resin, or a composite thereof may be used. Further, a single wire or twisted wires may be used.

The wire 22 wound on the pulley 20 is guided to an insertion passage 2a of the proximal end-side tube member 2 via a gap between the outer circumferential surface 16a and the inner circumferential surface of the through-hole 15a. The wire 22 from the insertion passage 2a is inserted and distributed into a tubular member 24, which has approximately the same inner diameter as the outer diameter of the wire 22, in which the wire 22 is slidably held, and which has flexibility without a change in axial length caused by forward and backward movement of the wire 22, and extends toward the proximal end side of the proximal end-side tube member 2 as shown in FIG. 5.

As to a configuration and material of the tubular member 24, for example, a coil pipe made by densely winding a metal wire, a pipe formed of a superelastic alloy, a tube formed of a synthetic resin, or the like are suitable. Among these, any one may be applied.

Further, the wire 23 wound on the pulley 21 is guided to the insertion passage 2a of the proximal end-side tube member 2 via a gap between the outer circumferential surface 19b and the inner circumferential surface of the through-hole 16c. Like the wire 22, the wire 23 from the insertion passage 2a is inserted and distributed into the tubular member 24, and extends toward the proximal end side of the proximal end-side tube member 2.

The wires 22 and 23 inserted into the tubular member 24 are, for instance, collected and inserted into a flexible pipe 25 formed of a synthetic resin or a rubber on the outside of the proximal end-side tube member 2, and are guided to the driving part 51 outside the proximal end-side tube member 2.

As shown in FIG. 5, the driving part 51 includes a drive system base 26 and a drive controller 32.

The drive system base 26 includes a support plate 26a that is supported approximately in a horizontal direction by a plurality of legs 26c.

The support plate 26a is provided with two tubular member fixing plates 26b on an upper surface thereof which fix an end of each tubular member 24 and into which the wires 22 and 23 are inserted in a horizontal direction.

To move the wires 22 and 23 forward and backward, motors 27 and 29 configured of, for example, stepping motors or servomotors are fixed at a lower surface side of the support plate 26a in a state in which the rotary shafts 27a and 29a protrude toward the upper surface side of the support plate 26a approximately in a vertical direction.

Driving pulleys 28 and 30 are fixed to the rotary shafts 27a and 29a, and are wound with the wires 22 and 23 inserted into the tubular member fixing plates 26b.

Power lines and signal lines of the motors 27 and 29 are collected on a cable 31 at an end of the support plate 26a, and are electrically connected to the drive controller 32 that controls rotation of the motors 27 and 29.

The drive controller 32 is configured of a computer having, for example, a central processing unit (CPU), a memory, an input/output interface, an external storage device, and so on. In the present embodiment, a manipulator 33 having a joystick 33a as a manipulating device for inputting a tilting amount and direction of the distal end-side tube member 4 is connected to the drive controller 32.

The joystick 33a may input the tilting direction of the distal end-side tube member 4 based on a direction inclined by manipulation, and the tilting amount depending on an inclined amount.

The drive controller 32 executes a proper control program. Thereby, the drive controller 32 analyzes manipulation input of the manipulator 33 which is generated by the manipulation of the joystick 33a, and calculates the tilting direction and amount of the distal end-side tube member 4. The drive controller 32 further calculates rotating amounts of the motors 27 and 29 based on the calculated tilting direction and amount, and thus generates control signals based on these rotating amounts. The control signals can be sent to the motors 27 and 29 via the cable 31.

With this configuration, the wires 22 and 23 inserted into the tubular members 24 are connected to the pulleys 20 and 21, and constitute a driving force transfer unit that undergoes remote manipulation to transfer a driving force from the outside of the proximal end-side tube member 2.

Next, an operation of the overcoat tube 90 of the present embodiment will be described.

FIG. 7 is a schematic explanatory view showing an operation of the overcoat tube according to the second embodiment of the present invention when viewed from the top (arrow D of FIG. 6).

When the joystick 33a is manipulated, the drive controller 32 calculates a tilting direction and amount of the distal end-side tube member 4 based on an inclined direction and amount of the joystick 33a. Depending on the calculated results, control signals based on rotating amounts of the motors 27 and 29 are sent to the motors 27 and 29.

For example, as shown in FIG. 7, when the distal end-side tube member 4 is tilted around the pivot shafts 17, only the motor 27 is rotated as shown in FIG. 5. Thereby, the driving pulley 28 is, for instance, rotated in a shown clockwise direction when viewed from the upper surface side of the support plate 26a. A driving force generated by the rotation of the driving pulley 28 is transferred to the pulley 20 via the wire 22 moving forward and backward in the tubular members 24. For this reason, as shown in FIG. 7, the pulley 20 is rotated in a clockwise direction shown in FIG. 7, and thus the pivot shafts 17 fixed to the pulley 20 and the intermediate case member 16 are rotated in the same direction.

As a result, the inner case 19 coupled to the intermediate case member 16 is also rotated in the clockwise direction shown in FIG. 7, and the distal end-side tube member 4 is tilted in a direction in line with the y axis via the connecting pipe part 4f connected to the inner case 19.

Similarly, when the motor 29 is rotated, a driving force generated by rotation of the driving pulley 30 is transferred to the pulley 21 via the wire 23 moving forward and backward in the tubular members 24. For this reason, the pivot shafts 18 fixed to the pulley 21 and the inner case 19 are rotated, the distal end-side tube member 4 is tilted in a direction perpendicular to the y axis within a plane, which is perpendicular to the distal end-side tube member central axis A₄, via the connecting pipe part 4f connected to the inner case 19.

Since the pivot shafts 17 and 18 are perpendicular to the pivotal center O, the pivoting motions of the two axial directions are not influenced by each other. For this reason, the distal end-side tube member 4 can be tilted in directions of the x- and y-axial directions perpendicular to the pivotal center O, and in an arbitrary axial direction passing through the pivotal center O within the xy plane.

In this manner, according to the present embodiment, the intermediate coupler 50 includes the pulleys 20 and 21 as drive mechanisms which are fixed to the intermediate case member 16 (movable member in the joint structure) that is a driving target and to the inner case 19 (driven part) and which are pivotably supported on the shafts (pivot shafts 17 and 18) that are perpendicular to the proximal end-side tube member central axis A₂ that is the tilting central axis, and the wires 22 and 23 as the driving force transfer units which are wound on the pulleys 20 and 21. Accordingly, by moving the wires 22 and 23 forward and backward to rotate the pulleys 20 and 21, the tilting of the distal end-side tube member 4 can be remotely manipulated from the outside of the proximal end-side tube member 2.

Furthermore, the overcoat tube 90 is configured so that the motors 27 and 29 that are driving force supply units supplying the driving force to the wires 22 and 23 are installed outside the proximal end-side tube member 2. For this reason, in comparison with a case of manually manipulating the wires 22 and 23, the tilting motion can be smoothly performed.

Further, like the first embodiment, the intermediate coupler 50 has the joint structure, so that it can be tilted around the pivotal center O at one joint. For this reason, for example, in comparison with a case in which the same tilting angle is obtained by a multiple joint bending mechanism in which a plurality of joint rings are pivotably coupled, an axial length can be shortened, and a length of the overcoat tube 90 can be shortened.

Further, similar to the first embodiment, the intermediate coupler 50 is provided to couple the distal end-side tube member 4 to the proximal end-side tube member 2 so that the distal end-side tube member 4 is capable of being tilted in the two axial directions centered on the pivotal center O with respect to the proximal end-side tube member 2, and simultaneously to communicate the insertion passages 4a and 2a of the distal and proximal end-side tube members 4 and 2 with each other. For this reason, the surgical space S can be widely secured even for the treatment target 104a located in shallow position from the body surface 102 by simple manipulation.

### Third Modified Example

Next, a modified example (third modified example) of the present embodiment will be described.

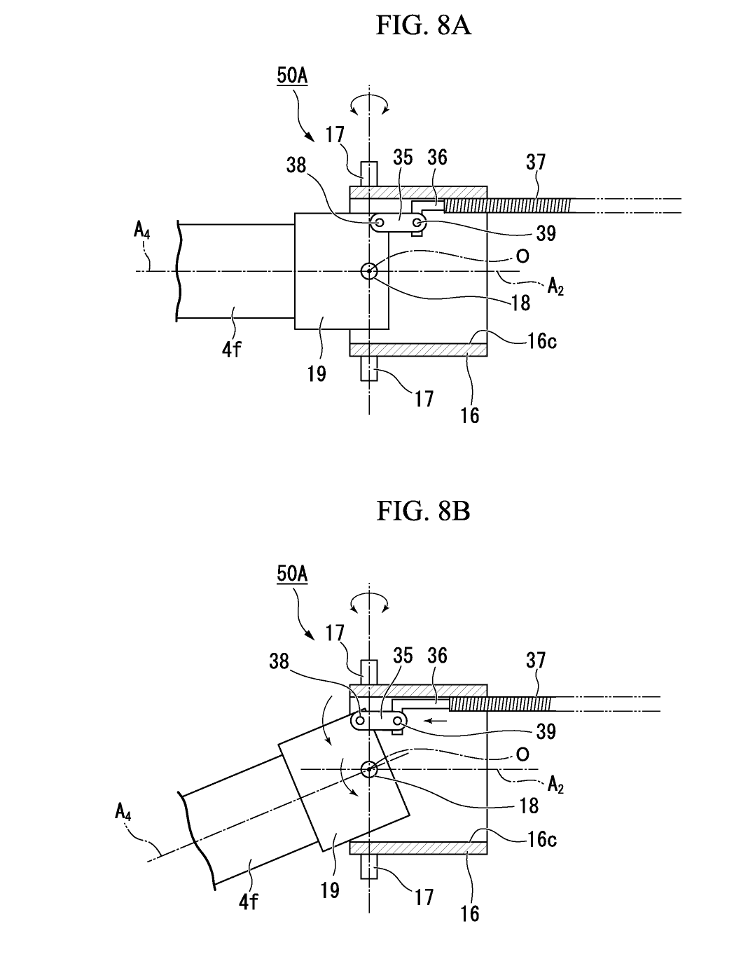

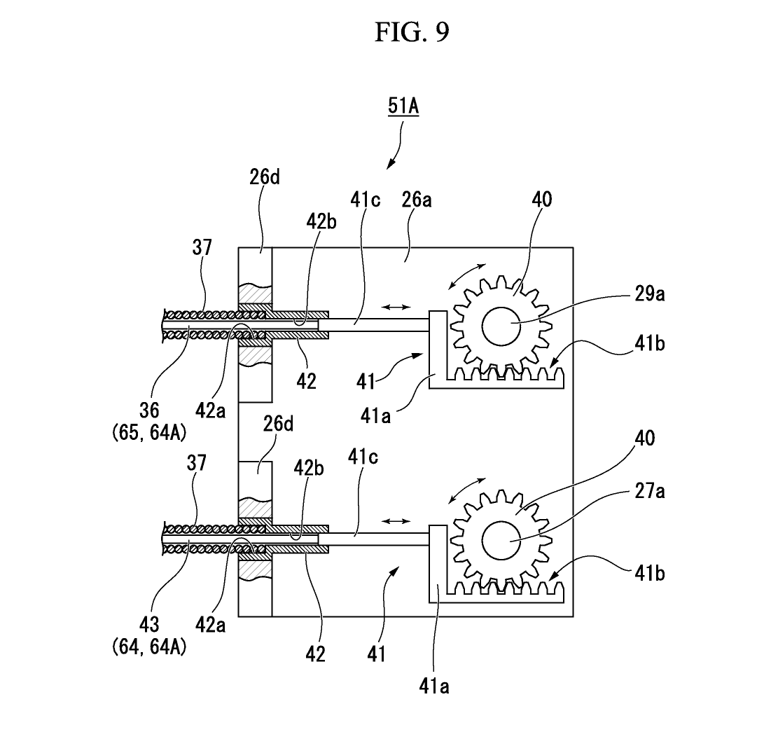

FIG. 8A is a schematic partial cross-sectional view showing a configuration of an intermediate coupler according to a modified example of the second embodiment of the present invention (third modified example). FIG. 8B is a schematic partial cross-sectional view showing a tilting state of the intermediate coupler according to the modified example of the second embodiment of the present invention (third modified example). FIG. 9 is a schematic plan view showing a configuration of a driving part according to the modified example of the second embodiment of the present invention (third modified example).

The present modified example includes an intermediate coupler 50A and a driving part 51A, in place of the intermediate coupler 50 and the driving part 51 of the overcoat tube 90 of the second embodiment.

The intermediate coupler 50A employs a link mechanism as a drive mechanism. As shown in FIG. 8A, the intermediate coupler 50A includes a link member 35 and a drive rod 36 in place of the pulley 21 and the wire 23 of the intermediate coupler 50 of the second embodiment which adopt the inner case 19 as the driving target. Similarly, in place of the pulley 20 and the wire 22 of the intermediate coupler 50 which adopt the intermediate case member 16 as the driving target, the intermediate coupler 50A includes a link member (not shown) similar to the link member 35, and a drive rod 43 (see FIG. 9) that is different only in length from the drive rod 36. However, since these components are different only in the driving targets, structures thereof can be easily understood, and so descriptions thereof will be omitted.

The link member 35 is a rod-like member that transfers a moment of force rotating the pivot shafts 18 to the inner case 19 that is the driving target. The link member 35 is configured so that one end side thereof is pivotably coupled to the inner case 19 via a rotating fulcrum 38 that is provided parallel to the pivot shafts 18 on an outer circumference of the inner case 19.

The other end side of the link member 35 is pivotably coupled to a distal end side of the drive rod 36 via a rotating fulcrum 39 parallel to the pivot shafts 18.

The drive rod 36 is a rod-like member that is disposed adjacent to an inner circumferential surface of a through-hole 16c capable of moving forward and backward in an axial direction. The drive rod 36 is inserted into a tubular member 37 so as not to be buckled when moving forward and backward, and is guided to an insertion passage 2a of a proximal end-side tube member 2.

As a material for the drive rod 36, a material such as a metal or a synthetic resin having flexibility may be employed.

The tubular member 37 has approximately the same inner diameter as the outer diameter of the drive rod 36, slidably holds the drive rod 36 therein, and has flexibility without a change in axial length caused by forward and backward movement of the drive rod 36. The tubular member 37 may employ a configuration and material similar to those of the tubular member 24 of the first embodiment.

The drive rod 36 is inserted and distributed into the tubular member 37 in the insertion passage 2a. Thus, like the tubular member 24 in the overcoat tube 90, the tubular member 37 is collected with another tubular member 37 into which the drive rod 43 that adopts the intermediate case member 16 as a driving target is inserted, and the collected tubular members 37 are inserted into a flexible pipe 25, and are guided to the driving part 51A outside the proximal end-side tube member 2.

As shown in FIG. 9, the driving part 51A includes a pair of tubular member fixing plates 26d, a pair of pinion gears 40, and a pair of driving members 41 in place of the tubular member fixing plates 26b and the driving pulleys 28 and 29 of the driving part 51 of the second embodiment.

Each tubular member fixing plate 26d is a member that fixes a fixing member 42, to which the other end of the tubular member 37 into which the drive rod 36 (43) is inserted is fixed, to a support plate 26a. The tubular member fixing plate 26d is fixed to the support plate 26a with the fixing member 42 held from an outer circumference side thereof.

As shown in FIG. 9, the fixing member 42 is a tubular member that includes a tubular member mounting hole 42a into which the other end of the tubular member 37 is inserted and fixed by, for instance, bonding or soldering, and a drive rod guide hole 42b that has a smaller inner diameter than an inner diameter of the tubular member mounting hole 42a and passes therethrough from a bottom of the tubular member mounting hole 42a in an axial direction.

Each pinion gear 40 supplies a driving force to the drive rod 36 (43). As such, the pinion gear 40 is fixed to a rotary shaft 29a (27a) of a motor 29 (27) fixed to the support plate 26a.

Each driving member 41 converts rotating motion of each pinion gear 40 into linear motion, and transfers the linear motion to the drive rod 36 (43). The driving member 41 is configured of a drive shaft 41c whose front end is connected to the other end side of the drive rod 36 (43) and is inserted into the drive rod guide hole 42b, and a drive block 41a that is connected to a rear end of the drive shaft 41c and has an L shape when viewed from the top. The drive shaft 41c has an outer diameter that is slightly smaller than an inner diameter of the drive rod guide hole 42b, and is allowed to move forward and backward in the drive rod guide hole 42b in an axial direction.

A region of the drive block 41a which extends parallel to the drive shaft 41c is provided with a rack 41b that is meshed with the pinion gear 40 and converts the rotating motion of the pinion gear 40 into the linear motion.

According to the driving part 51A of the present modified example, similar to the driving part 51, when a manipulator 33 is manipulated, the motors 27 and 29 are rotated based on an amount of manipulation, and the racks 41b are linearly driven by the pinion gears 40 fixed to the rotary shafts 27a and 29a.

For this reason, the drive shafts 41c of the driving members 41 move forward or backward in the drive rod guide holes 42b in an axial direction, and the drive rods 36 and 43 fixed to the drive shafts 41c move forward or backward in an axial direction.

For example, when the drive rod 36 moves toward the distal end side thereof in an axial direction, the distal end of the drive rod 36 moves toward the distal end side of the intermediate case member 16 as shown in FIG. 8B, and the moment of force rotating the pivot shafts 18 is applied to the inner case 19 coupled via the link member 35. For this reason, the inner case 19 and the connecting pipe part 4f connected to the inner case 19 pivot around the pivot shafts 18 in a shown counterclockwise direction. As a result, the distal end-side tube member 4 is tilted in the shown counterclockwise direction with respect to the proximal end-side tube member central axis A₂.

Similarly, the motor 27 is rotated, thereby allowing the intermediate case member 16 to pivot around the pivot shafts 17. Thus, the distal end-side tube member 4 coupled to the intermediate case member 16 via the inner case 19 can be tilted around the pivot shafts 17.

In this way, the present modified example is an example in which, even when the link mechanism as the drive mechanism and the drive rods 36 and 43 that are the rod-like member are used, the distal end-side tube member 4 can be tilted, as in the case in which the pulleys and the wires are used.

In the case of the present modified example, the driving force transfer units are formed by the drive rods 36 and 43. Accordingly, as in the case in which the wires 22 and 23 are employed, winding the wires on the pulleys 20 and 21 and adjusting tension of the wires are not necessary, and thus assembly and maintenance become easier.

### Third Embodiment

An overcoat tube according to a third embodiment of the present invention will be described.

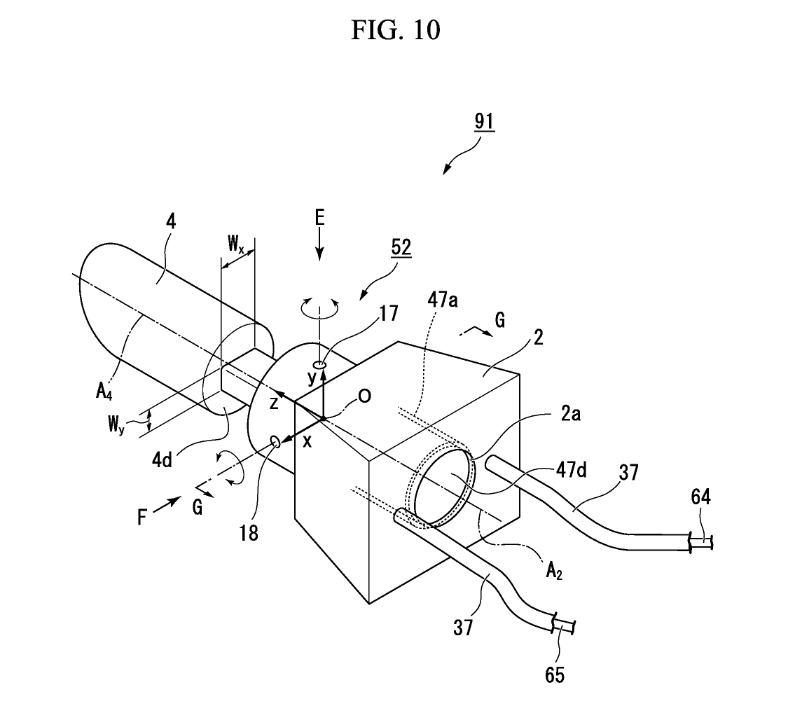

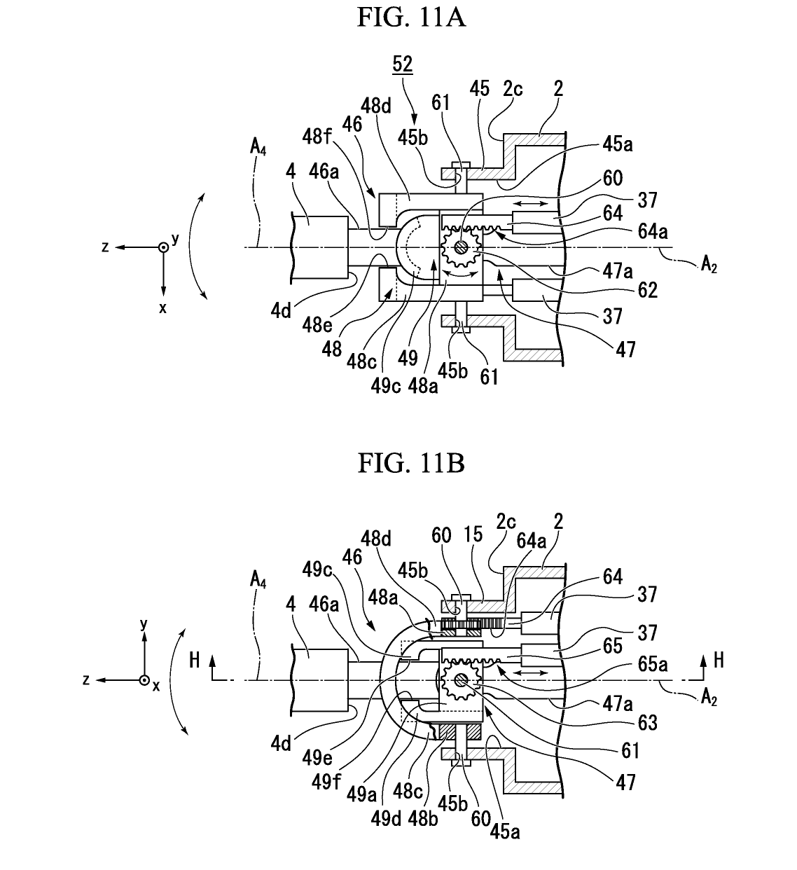

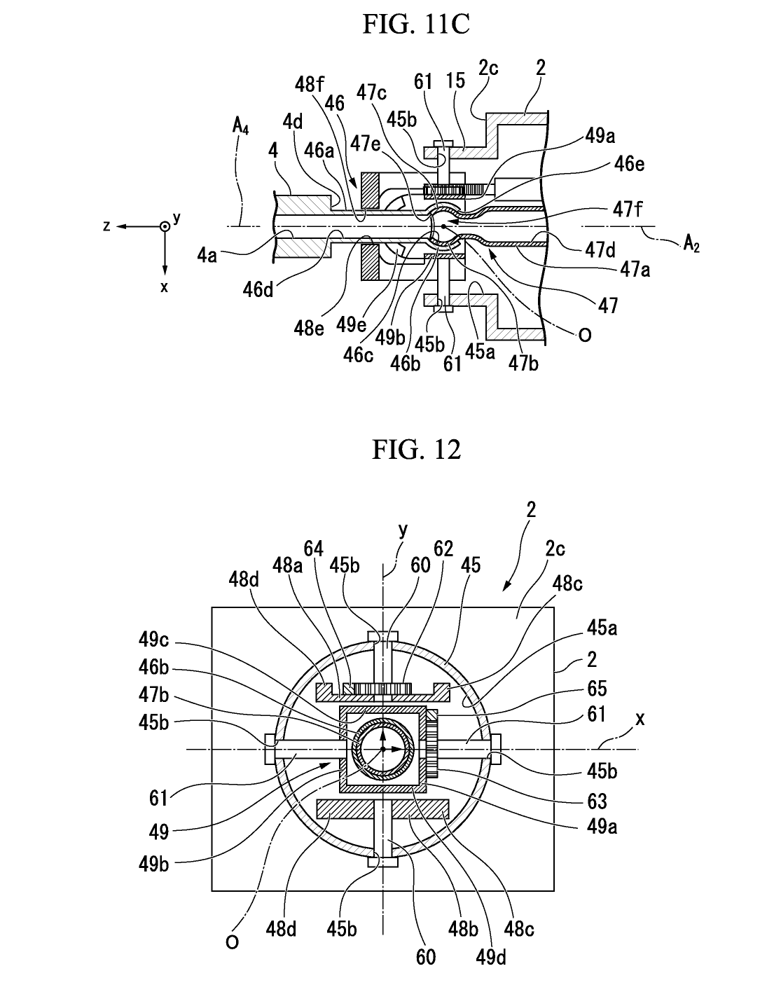

FIG. 10 is a schematic perspective view showing a configuration of a main part in an overcoat tube according to a third embodiment of the present invention. FIG. 11A is a partial cross-sectional view when viewed from arrow E of FIG. 10. FIG. 11B is a partial cross-sectional view when viewed from arrow F of FIG. 10. FIG. 11C is a cross-sectional view taken along line H-H of FIG. 11B. FIG. 12 is a cross-sectional view taken along line G-G of FIG. 10.

As shown in FIG. 10, the overcoat tube 91 of the present embodiment includes an intermediate coupler 52 and a driving part 51A (see FIG. 9) in place of the intermediate coupler 50 and the driving part 51 of the overcoat tube 90 of the second embodiment. Like the overcoat tube 90 of the second embodiment, the overcoat tube 91 is configured so that a distal end-side tube member 4 can be tilted in two axial directions centered on a pivotal center O on a proximal end-side tube member central axis A₂ with respect to the proximal end-side tube member central axis A₂ of a proximal end-side tube member 2. The following description will focus on differences between the present embodiment and the second embodiment.

As shown in FIGS. 11A, 11B, 11C, and 12, the intermediate coupler 52 includes a female coupler 46, a male coupler 47, a tubular part 45, a first movement restriction member 48 (movement restriction member), a pinion gear 62 (drive mechanism), a second movement restriction member 49 (movement restriction member), and a pinion gear (drive mechanism) 63.

For simplicity, unless otherwise mentioned, the following description will be made regarding a positional relationship when a distal end-side tube member central axis A₄ of the distal end-side tube member 4 is aligned with the proximal end-side tube member central axis A₂, i.e., is not tilted. Further, the following description may be made regarding a relative positional relationship using an xyz coordinate system that consists of a z axis (where a negative direction of the z axis is set to a proximal end side of the proximal end-side tube member 2) aligned with the proximal end-side tube member central axis A₂, and x and y axes perpendicular to the z axis using the pivotal center O as an origin.

The female coupler 46 includes a tubular part (driven part) 46a connected to a proximal end face 4d of the distal end-side tube member 4, and a partial spherical shell-shaped female joint part 46b connected to a proximal end side of the tubular part 46a.

In the tubular part 46a, a through-hole 46d having an inner diameter that is equal to that of an insertion passage 4a on a proximal end side of the distal end-side tube member 4 is provided.

Further, in the present embodiment, an external form of the tubular part 46a is a tetragonal prism shape in which the distal end-side tube member central axis A₄ is set as a central axis, and x- and y-axial widths are set to Wₓ and W_{y}, respectively.

The female joint part 46b is provided with a concave engaging surface 46c on an inner side thereof which is a partial spherical surface having an inner diameter greater than that of the through-hole 46d.

Further, the female joint part 46b is provided with a circular hole-shaped opening 46e on a proximal end side thereof which is greater than the inner diameter of the through-hole 46d and which is centered on the distal end-side tube member central axis A₄.

The male coupler 47 includes a tubular part (support) 47a that has a through-hole 47d communicating with an insertion passage 2a of the proximal end-side tube member 2 in the center thereof, and a partial spherical shell-shaped male joint part 47b connected to a distal end side of the tubular part 47a.

As shown in FIG. 10, the tubular part 47a is disposed such that a central axis thereof is coincident with the proximal end-side tube member central axis A₂ of the proximal end-side tube member 2, and is connected with the proximal end-side tube member 2 in a state in which an inner circumferential surface of the through-hole 47d is aligned with that of the insertion passage 2a of the proximal end-side tube member 2.

As shown in FIG. 11C, an outer circumferential surface of the male joint part 47b is provided with a convex engaging surface 47c that is a partial spherical surface slidably fitted into the concave engaging surface 46c of the female coupler 46, and is disposed such that the center of the convex engaging surface 47c is coincident with the pivotal center O.

Further, in the male joint part 47b, a spherical cavity part 47f that communicates with the through-hole 47d of the tubular part 47a at a proximal end side thereof is provided.

In addition, the male joint part 47b is provided with a circular hole-shaped opening 47e on a distal end side thereof which is centered on the proximal end-side tube member central axis A₂, and whose inner diameter is greater than that of the through-hole 46d.

With this configuration, as shown in FIG. 11C, the male joint part 47b of the male coupler 47 is fitted into the female joint part 46b of the female coupler 46. Thereby, the female coupler 46 and the male coupler 47 are slidably engaged with each other by the concave engaging surface 46c and the convex engaging surface 47c.

For this reason, the male coupler 47 can freely pivot around the pivotal center O, which is a common curvature center of the concave engaging surface 46c and the convex engaging surface 47c, within a range in which the external form of the tubular part 47a is in contact with an inner edge of the opening 46e.

Thereby, the distal end-side tube member 4 connected to the female coupler 46 is coupled to be able to be tilted with respect to the proximal end-side tube member central axis A₂ using the pivotal center O as a tilting center.

In this way, like the female and male couplers 6 and 7 of the intermediate coupler 3 of the first embodiment, the female and male couplers 46 and 47 constitute a ball joint that can be tilted in two axial directions. The case in which the support is connected to the male joint part 47b and in which the driven part is connected to the female joint part 46b is taken by way of example.

As a material for the female and male couplers 46 and 47, a material similar to that for the female and male couplers 6 and 7 may be employed.

Further, the cavity part 47f and the through-hole 47d inside the male coupler 47 communicate with the insertion passage 2a. Also, however the male coupler 47 is pivoted, the opening 47e is open toward the through-hole 46d of the female coupler 46. For this reason, the insertion passage 4a communicates with the insertion passage 2a via the through-hole 46d, the cavity part 47f, and the through-hole 47d. Thereby, in the overcoat tube 91, an insertion passage that runs through in an axial direction is provided.

The tubular part 45 is an annular protrusion part that protrudes from a stepped part 2c of the proximal end-side tube member 2 at the distal end side of the proximal end-side tube member 2 to have the same axis as the proximal end-side tube member central axis A₂, and in which a through-hole 45a passing therethrough is provided.

As shown in FIG. 12, an inner diameter of the through-hole 45a is set to be greater than an outer diameter of the first movement restriction member 48 which will be described below.

The tubular part 45 is provided with four holes 45b in a lateral surface thereof which pass therethrough in a thickness-wise direction in line with the x-axial direction (horizontal direction of FIG. 12) and the y-axial direction (vertical direction of FIG. 12) within a plane that passes through the pivotal center O of the female and male couplers 46 and 47 and that is perpendicular to the proximal end-side tube member central axis A₂. In the present embodiment, the x and y axes are disposed in the directions that approximately run along long and short sides of a rectangular external form of the stepped part 2c.

The first movement restriction member 48 restricts x-axial positions of lateral portions of the tubular part 46a at positions spaced apart from the pivotal center O, which is the tilting center of the female and male couplers 46 and 47, in the positive direction of the z axis.

In the present embodiment, as shown in FIGS. 11A, 11B, 11C, and 12, the first movement restriction member 48 is configured of a frame member that includes U-shaped arm parts 48c and 48d that are disposed apart in the x-axial direction in a U-shaped external form when viewed from the side (arrow F) and that are provided in a plane-symmetrical shape with respect to a central plane of the y-axial direction, tabular bridge parts 48a and 48b that connect U-shaped openings to each other at proximal end sides of the U-shaped arm parts 48c and 48d in the x-axial direction, and a pair of pivot shafts 60 that are erected toward the positive-directional side of the y axis (upper side shown in FIG. 12) and the negative-directional side of the y axis (lower side shown in FIG. 12) at middle portions of a lengthwise direction (x-axial direction) of the bridge parts 48a and 48b and that have the same axis as the y axis.

As shown in FIGS. 11A and 11C, U-shaped curved portions of the U-shaped arm parts 48c and 48d become x-axial thick walls, compared to U-shaped linear portions, and are provided with holding surface parts 48e and 48f on inner sides thereof. An x-axial distance between the holding surface parts 48e and 48f is approximately equal to the x-axial width Wₓ of the tubular part 46a, and thus the tubular part 46a can be slidably sandwiched between the holding surface parts 48e and 48f.

In the present embodiment, the first movement restriction member 48 is sufficient if it can restrict the position in line with the x-axial direction of the tubular part 46a, with no need to restrict an orientation of the tubular part 46a. For this reason, contact widths of the holding surface parts 48f and 48e coming into contact with the tubular part 46a may also be narrow. For example, each of the holding surface parts 48e and 48f may have a shape in which it comes into line contact with the tubular part 46a, such as a shape in which it has a convex arcuate cross section in the opposite directions.

The pivot shafts 60 are pivotably coupled to the pair of holes 45b that are opposite in the y-axial direction of the tubular part 45. In this case, the holding surface parts 48e and 48f are disposed in a plane-symmetrical positional relationship with respect to a plane including the proximal end-side tube member central axis A₂ and the y axis.

The pinion gear 62 is a drive mechanism for driving the first movement restriction member 48, and is fixed to a root side of the pivot shaft 60 on the bridge part 48a.

A drive rod 64 is disposed beside the pinion gear 62. The drive rod 64 is provided with a rack 64a meshed with the pinion gear 62, and is installed to be able to move forward and backward in the z-axial direction.

The drive rod 64 constitutes a driving force transfer unit that is remotely manipulated from the outside of the proximal end-side tube member 2 and thus transfers a driving force to the pinion gear 62. Like the drive rod 36 of the modified example of the second embodiment (third modified example), the drive rod 64 is inserted into the tubular member 37 and is distributed into the proximal end-side tube member 2. Then, as shown in FIG. 10, the drive rod 64 extends outwardly from the proximal end side of the proximal end-side tube member 2.

The second movement restriction member 49 restricts y-axial positions of the lateral portions of the tubular part 46a at positions spaced apart from the pivotal center O in the positive direction of the z axis.

In the present embodiment, as shown in FIGS. 11A, 11B, 11C, and 12, the second movement restriction member 49 is configured of a frame member that includes round-point arm parts 49c and 49d in which tabular members, whose distal end sides (positive-directional sides of the z axis) are rounded in an external form when viewed from the top (arrow E), are disposed apart in the y-axial direction and which are provided in a plane-symmetrical shape with respect to a central plane of the x-axial direction, tabular bridge parts 49a and 49b that connect proximal ends of the round-point arm parts 49c and 49d to each other in the y-axial direction, and a pair of pivot shafts 61 that are erected toward the positive-directional side of the x axis (right side shown in FIG. 12) and the negative-directional side of the x axis (left side shown in FIG. 12) at middle portions of a lengthwise direction (y-axial direction) of the bridge parts 49a and 49b and that have the same axis as the x axis.

The second movement restriction member 49 has external dimensions in which an x-axial outer width of each of the round-point arm parts 49c and 49d is smaller than an inner width of each of the U-shaped arm parts 48c and 48d of the first movement restriction member 48, and in which a y-axial outer width of each of the round-point arm parts 49c and 49d is smaller than an x-axial opposite interval between the U-shaped arm parts 48c and 48d of the first movement restriction member 48. The distal ends of the round-point arm parts 49c and 49d have such a size as not to interfere with the inner sides of the U-shaped arm parts 48c and 48d of the first movement restriction member 48 when the second movement restriction member 49 pivots.

Thereby, a movable region of the second movement restriction member 49 is disposed within that of the first movement restriction member 48. Here, the movable region refers to an entire space region in which the first and second movement restriction members 48 and 49 sweep when pivoting.

As shown in FIG. 11B, the distal end sides of the round-point arm parts 49c and 49d are provided with arcuate thick-walled parts along rounded outer edges, and holding surface parts 49e and 49f on inner sides thereof. A y-axial distance between the holding surface parts 49e and 49f is approximately equal to the y-axial width W_{y} of the tubular part 46a, and thus the tubular part 46a can be slidably sandwiched between the holding surface parts 49e and 49f.

In the present embodiment, the second movement restriction member 49 is sufficient if it can restrict the position in line with the y-axial direction of the tubular part 46a, with no need to restrict an orientation of the tubular part 46a. For this reason, contact widths of the holding surface parts 49f and 49e coming into contact with the tubular part 46a may also be narrow. For example, each of the holding surface parts 49e and 49f may have a shape in which it comes into line contact with the tubular part 46a, such as a shape in which it has a convex arcuate cross section in the opposite directions.

The pivot shafts 61 are pivotably coupled to the pair of holes 45b that are opposite in the x-axial direction of the tubular part 45. In this case, the holding surface parts 49e and 49f are disposed in a plane-symmetrical positional relationship with respect to a plane including the proximal end-side tube member central axis A₂ and the x axis.

The pinion gear 63 is a drive mechanism for driving the second movement restriction member 49, and is fixed to a root side of the pivot shaft 61 on the bridge part 49a.

A drive rod 65 is disposed beside the pinion gear 63. The drive rod 65 is provided with a rack 65a meshed with the pinion gear 63, and is installed to be able to move forward and backward in the z-axial direction.

The drive rod 65 constitutes a driving force transfer unit that is remotely manipulated from the outside of the proximal end-side tube member 2 and thus transfers a driving force to the pinion gear 63. The drive rod 65 is configured like the drive rod 64, is inserted into the tubular member 37 and is distributed into the proximal end-side tube member 2. Then, as shown in FIG. 10, the drive rod 65 extends outwardly from the proximal end side of the proximal end-side tube member 2.

The tubular members 37 into which the drive rods 64 and 65 are inserted are connected to the driving part 51A of the modified example of the second embodiment (third modified example) as shown in FIG. 9.

In this case, the drive rods 64 and 65 are coupled to driving members 41 of the driving part 51A, and are driven to move forward and backward in the tubular members 37 by the motors 27 and 29, respectively.

Next, an operation of the overcoat tube 91 of the present embodiment will be described focusing on points different from the second embodiment and its modified example.

According to the present embodiment, when the manipulator 33 is manipulated, the motors 27 and 29 of the driving part 51A are rotated based on an amount of manipulation, and the drive rods 64 and 65 fixed to the drive shafts 41c move forward or backward in the axial direction depending on rotation amounts of the motors 27 and 29, similar to the modified example of the second embodiment.

For example, when the drive rod 64 moves toward the distal end side thereof in an axial direction, the rack 64a of the drive rod 64 moves in the positive direction of the z axis, and the pinion gear 62 is rotated in a counterclockwise direction shown in FIG. 11A. Thereby, the pivot shaft 60 to which the pinion gear 62 is fixed is rotatably driven, and the first movement restriction member 48 is rotated about the pivot shaft 60 in the shown counterclockwise direction. As a result, the tubular part 46a sandwiched between the holding surface parts 48e and 48f at a position spaced apart from the pivot shaft 60 is tilted around the y axis centered on the pivotal center O with respect to the proximal end-side tube member central axis A₂. For this reason, the distal end-side tube member 4 connected to the tubular part 46a is also tilted in a similar way. In this case, x-axial positions of lateral portions of the tubular part 46a are restricted by the holding surface parts 49e and 49f of the second movement restriction member 49.

Further, even when the first movement restriction member 48 is displaced, the second movement restriction member 49 is housed inside the movable region of the first movement restriction member 48, and thus does not interfere with the first movement restriction member 48.

When the drive rod 64 moves backward to the proximal end side thereof in an axial direction, a tilting motion opposite to this tilting motion is performed.

Similarly, for example, when the drive rod 65 moves toward the distal end side thereof in an axial direction, the rack 65a of the drive rod 65 moves in the positive direction of the z axis, and the pinion gear 63 is rotated in a counterclockwise direction shown in FIG. 11B. Thereby, the pivot shaft 61 to which the pinion gear 63 is fixed is rotatably driven, and the second movement restriction member 49 is rotated about the pivot shaft 61 in the shown counterclockwise direction. As a result, the tubular part 46a sandwiched between the holding surface parts 49e and 49f at a position spaced apart from the pivot shaft 61 is tilted around the x axis centered on the pivotal center O with respect to the proximal end-side tube member central axis A₂. For this reason, the distal end-side tube member 4 connected to the tubular part 46a is also tilted in a similar way. For this reason, y-axial positions of lateral portions of the tubular part 46a are restricted by the holding surface parts 48e and 48f of the first movement restriction member 48.

Further, even when the second movement restriction member 49 is displaced, the second movement restriction member 49 does not interfere with the first movement restriction member 48 in a similar way.

When the drive rod 65 moves backward to the proximal end side thereof in an axial direction, a tilting motion opposite to this tilting motion is performed.

When the drive rods 64 and 65 move forward or backward at the same time, pivotal positions of the first and second movement restriction members 48 and 49 are positively decided based on the amounts of forward or backward movement. For this reason, the tubular part 46a is tilted in a direction in which the y-axial position restricted by the first movement restriction member 48, the x-axial position restricted by the second movement restriction member 49, and the pivotal center O are connected.

In this manner, the overcoat tube 91 of the present embodiment includes the first and second movement restriction members 48 and 49 which are pivotably coupled to the proximal end-side tube member 2 and which restrict the positions of the lateral portions of the tubular part 46a, which is the driven part, at the positions spaced apart from the tilting center of the joint structure, and the pinion gears 62 and 63 as the drive mechanisms which drive the first and second movement restriction members 48 and 49 to thereby tilt the tubular part 46a with respect to the tubular part 47a coupled to the proximal end-side tube member 2 that is the support. Thereby, the overcoat tube 91 can be tilted in the two axial directions centered on the pivotal center O, as in the second embodiment.

Here, the holding surface parts 48e and 48f of the first movement restriction member 48 (or the holding surface parts 49e and 49f of the second movement restriction member 49) constitute a slit in which the lateral portions of the driven part are sandwiched in a circumferential direction of a pivotal circle of the first movement restriction member 48 (or the second movement restriction member 49).

Accordingly, like the second embodiment, the intermediate coupler 52 can shorten the axial length thereof, and attempt to shorten the length of the overcoat tube 91, compared to, for instance, the case in which the same tilting angle is obtained by the multiple joint bending mechanism in which a plurality of joint rings are pivotably coupled.

Further, since the intermediate coupler 52 is provided, a surgical space can be widely secured even for a treatment target located in shallow position from a body surface by simple manipulation.

### Fourth Modified Example

Next, a modified example of the present embodiment (fourth modified example) will be described.

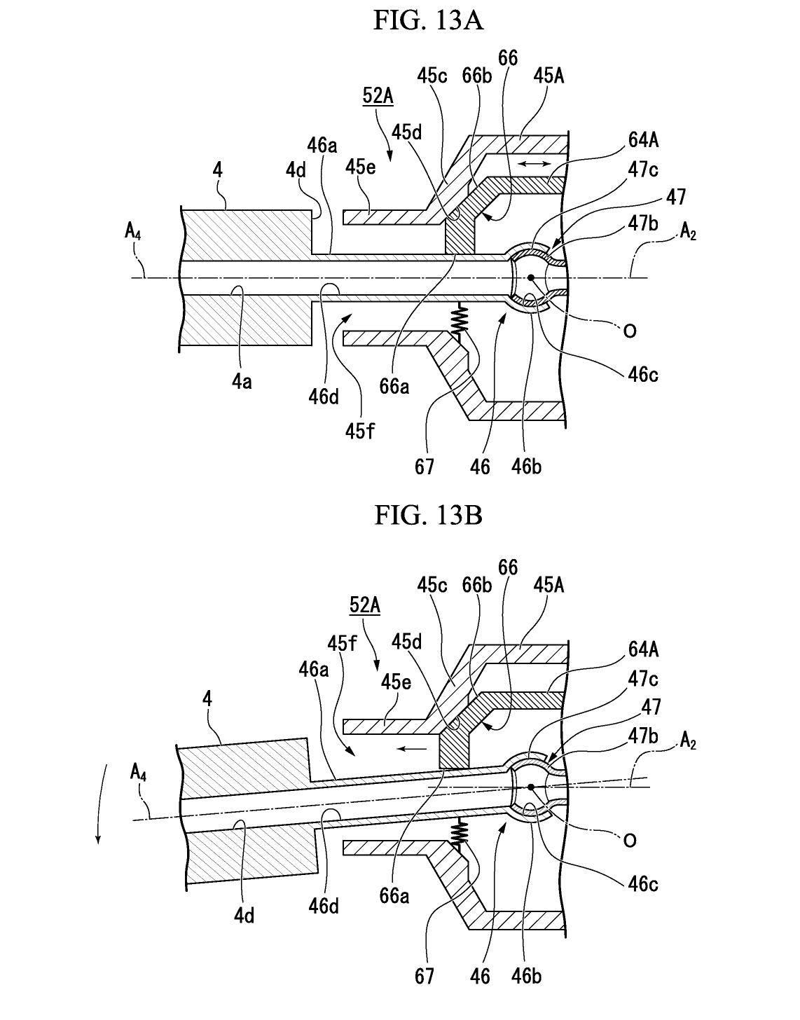

FIG. 13A is a schematic cross-sectional view showing a configuration of a main part in the intermediate coupler according to a modified example (fourth modified example) of the third embodiment of the present invention. FIG. 13B is a schematic cross-sectional view showing a tilting state of the intermediate coupler according to a modified example of the third embodiment (fourth modified example) of the present invention.

The present modified example includes an intermediate coupler 52A in place of the intermediate coupler 52 of the overcoat tube 91 of the third embodiment.

The intermediate coupler 52A is a modified example of the movement restriction members and the drive mechanisms of the intermediate coupler 52 of the third embodiment. As shown in FIG. 13A, the intermediate coupler 52A is configured such that the first and second movement restriction members 48 and 49, the pivot shafts 60 and 61, and the pinion gears 62 and 63 are removed from the configuration of the intermediate coupler 52 of the second embodiment, and the intermediate coupler 52A includes a tubular part 45A and drive rods 64A in place of the tubular part 45 and the drive rod 64 (65), and further includes an elastic member 67. The following description will focus on differences between the present modified example and the third embodiment.

The tubular part 45A includes a movement guide part 45c that is slightly tilted toward a radial inner side and a distal end side thereof at a distal end side of a cylindrical member from which the holes 45b of the tubular part 45 are removed, and a distal end annular part 45e that extends from an inner edge of the movement guide part 45c to the distal end side along a proximal end-side tube member central axis A₂, and is a tubular part whose diameter is reduced to the distal end side as a whole.

Pivotal centers O of a female coupler 46 and a male coupler 47 are disposed on a distal end-side tube member central axis A₄ in the tubular part 45A.

Further, the distal end annular part 45e has a distal end opening 45f whose diameter is smaller than that of the tubular part 45A and is greater than an external form of a through-hole 46d, and which has such a size as to be able to tilt the female coupler 46 with a tubular part 46a of the female coupler 46 inserted thereinto, similar to the third embodiment.

Further, the movement guide part 45c is provided with a conical movement guide surface 45d on an inner circumferential surface thereof which adopts the proximal end-side tube member central axis A₂ as a central axis, and whose diameter is reduced to the axial distal end side.

Each drive rod 64A includes a lateral-portion pressing part 66 in place of the rack 64a (65a) of the drive rod 64 (65) of the third embodiment. The two drive rods 64A are provided to correspond to the drive rods 64 and 65, are inserted into tubular members 37 similar to the drive rods 64 and 65, and are connected to a driving part 51A at an end of the proximal end side thereof (see FIG. 9).

The two drive rods 64A are similarly disposed along the x and y axes to be tilted in two axial directions.

The lateral-portion pressing part 66 is a bent part that is bent in two steps toward the proximal end-side tube member central axis A₂ at the distal end of the drive rods 64A, and includes a slide surface 66b that is inclined and bent at an inclined angle similar to that of a movement guide surface 45d and comes into slidable contact with the movement guide surface 45d, and a pressing surface 66a that extends from a distal end of the slide surface 66b along the x or y axis and comes into contact with a lateral portion of the tubular part 46a at a distal end of the extending direction.

The elastic member 67 biases one opposite the lateral portion of tubular part 46a with which each pressing surface 66a comes into contact from the movement guide part 45c with an elastic force. As the elastic member 67, for example, an arbitrary spring member such as a compression spring or a leaf spring, or an elastic member obtained by forming a rubber in an arbitrary shape such as a rod shape or a bellows shape may be employed.

With this configuration, when the drive rod 64A moves forward at a given position, the lateral-portion pressing part 66 is positioned by causing the slide surface 66b to come into close contact with the movement guide surface 45d, and the pressing surface 66a is disposed at a position spaced apart from the proximal end-side tube member central axis A₂ by a certain distance.

On the other hand, on the opposite side of the pressing surface 66a, the tubular part 46a biased by the elastic member 67 is pressed toward the pressing surface 66a, and thus the lateral portion of the tubular part 46a is positioned by the pressing surface 66a.

For example, in FIG. 13A, the tubular part 46a is positioned with a positional relationship in which the proximal end-side tube member central axis A₂ is aligned with the distal end-side tube member central axis A₄.

An operation of the intermediate coupler 52A of the present modified example will be described.

Like the third embodiment, when a manipulator 33 is driven to move the drive rod 64A forward or backward, the slide surface 66b of the lateral-portion pressing part 66 is displaced along the movement guide surface 45d. For this reason, a distance between the pressing surface 66a and the proximal end-side tube member central axis A₂ varies depending on an amount of axial movement of the drive rod 64A.

For example, as shown in FIG. 13B, when the drive rod 64A moves forward to the distal end side from the state of FIG. 13A, the lateral-portion pressing part 66 is displaced to the distal end side along the movement guide surface 45d. As a result, the distance between the pressing surface 66a and the proximal end-side tube member central axis A₂ is reduced, and the tubular part 46a is further pressed toward the proximal end-side tube member central axis A₂ (toward a shown lower side). As a result, the tubular part 46a pivots around the pivotal center O, and is tilted in a counterclockwise direction.

Similarly, when the drive rod 64A moves backward, the distance between the pressing surface 66a and the proximal end-side tube member central axis A₂ is increased, and the tubular part 46a biased by the elastic member 67 is displaced along with the pressing surface 66a with the lateral portion thereof kept in contact with the pressing surface 66a. For this reason, the tubular part 46a pivots around the pivotal center O, and is tilted in a clockwise direction.

A motion caused by the other drive rod 64A which is not shown is similar. Thereby, like the third embodiment, the distal end-side tube member 4 can be tilted in the two axial directions.

In this manner, the lateral-portion pressing part 66 of the present modified example comes into contact with the lateral surface of the driven part, and is provided to be able to move forward or backward in the axial direction of the proximal end-side tube member 2. The movement guide part 45c having the movement guide surface 45d changes a position in a direction perpendicular to the axial direction of the lateral-portion pressing part 66 based on an axial position of the lateral-portion pressing part 66.

For this reason, the lateral-portion pressing part 66 and the movement guide part 45c constitute a movement restriction member that is provided on the proximal end-side tube member 2 and restricts the positions of the lateral portions of the driven part at positions spaced apart from the tilting center of the joint structure.

Further, among them, the lateral-portion pressing part 66 is integrally provided on the drive rod 64A constituting a driving force transfer unit, like the drive rods 64 and 65.

According to the present modified example, as the movement restriction member, the movement guide part 45c fixed to the distal end-side tube member 4 and the lateral-portion pressing part 66 integrated into the drive rods 64A are used. For this reason, compared to the case in which the first and second movement restriction members 48 and 49 are used as in the third embodiment, a simple configuration can be obtained, and a space-saving configuration can be obtained.

### Fourth Embodiment

An overcoat tube according to a fourth embodiment of the present invention will be described.

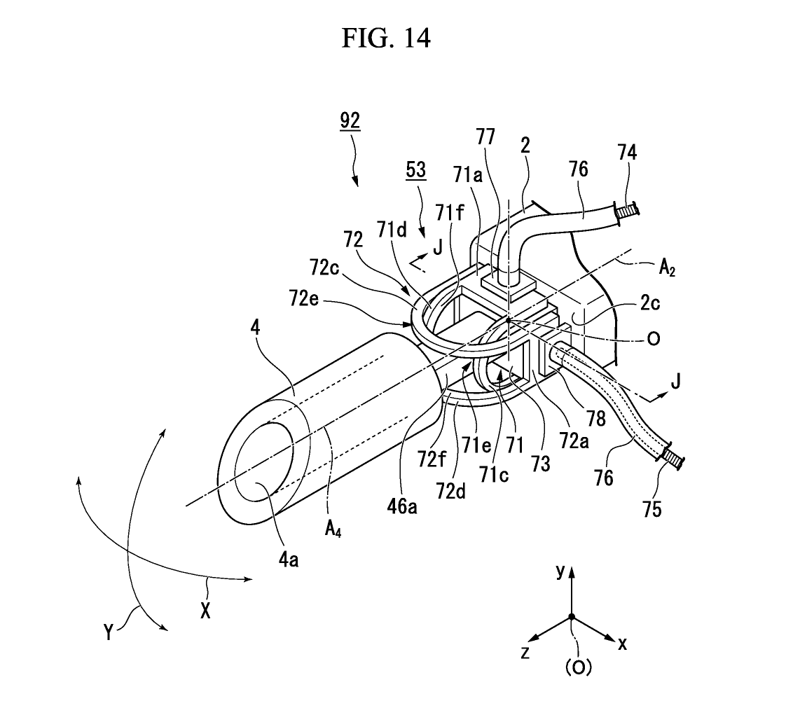

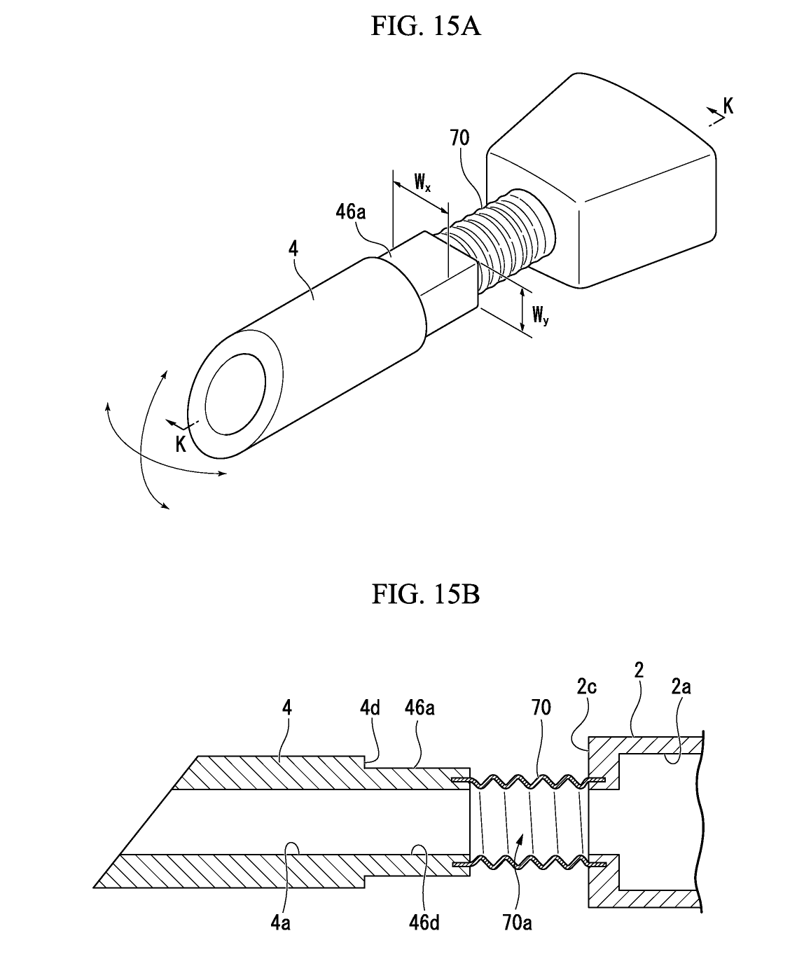

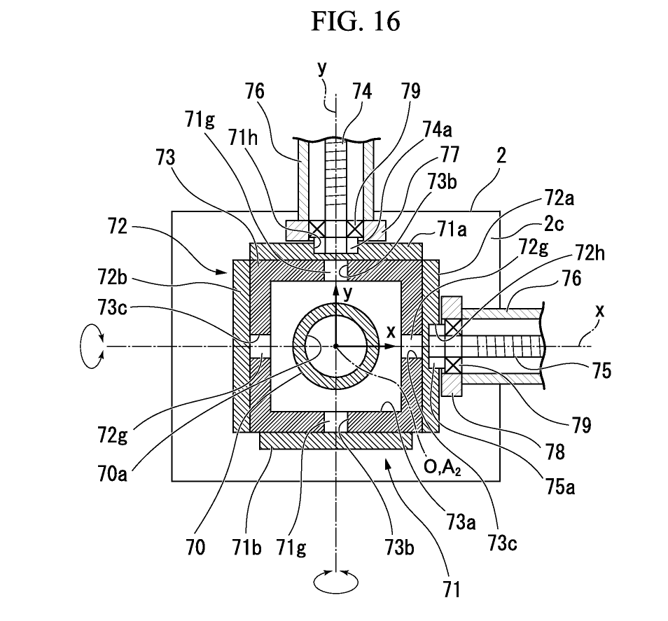

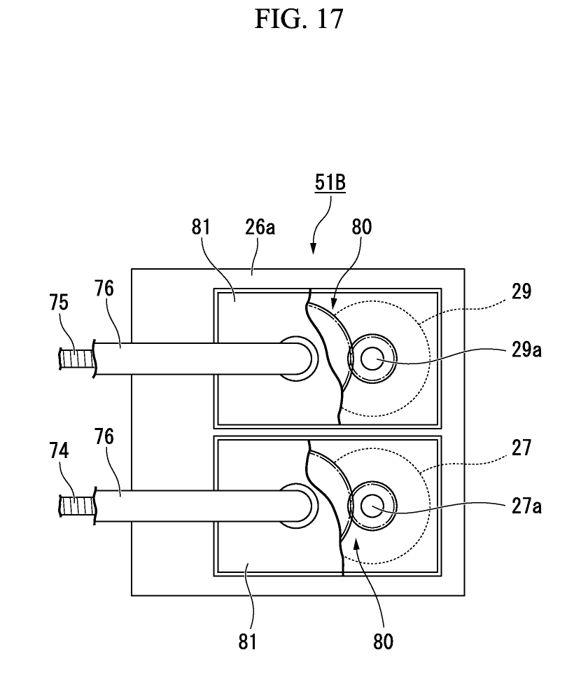

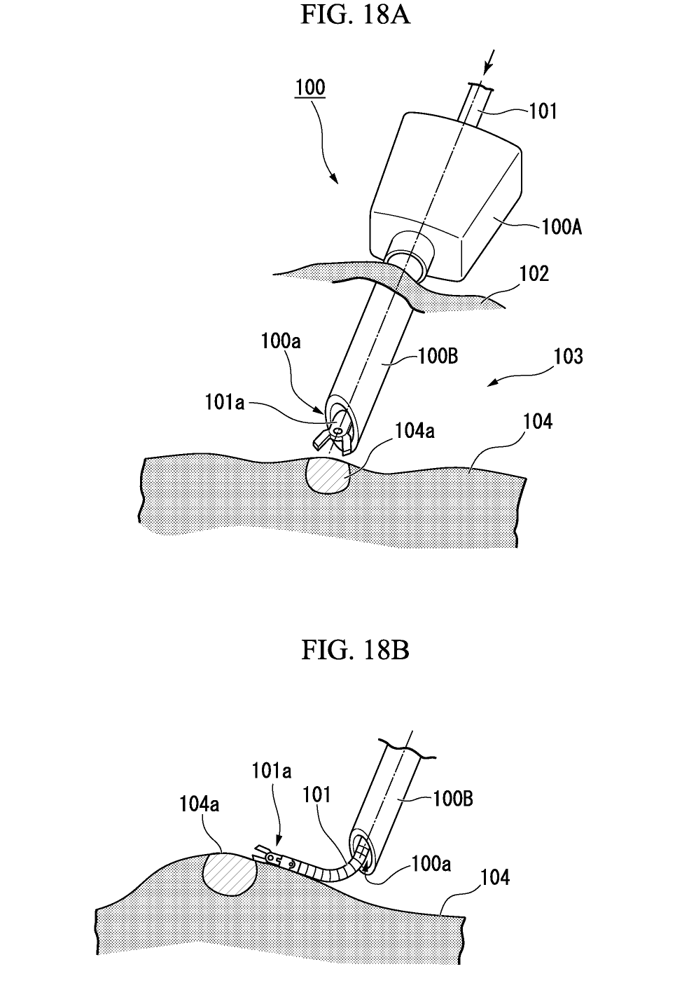

FIG. 14 is a schematic perspective view showing a configuration of a main part in an overcoat tube according to a fourth embodiment of the present invention. FIG. 15A is a schematic perspective view showing a configuration of an intermediate tube member in the overcoat tube according to the fourth embodiment of the present invention. FIG. 15B is a cross-sectional view taken along line K-K of FIG. 15A. FIG. 16 is a cross-sectional view taken along line J-J of FIG. 14. FIG. 17 is a schematic plan view showing a configuration of a driving part according to the fourth embodiment of the present invention. In FIG. 14, for a clearer view, positions of xyz axes to be depicted using a pivotal center O as an origin are depicted with slight displacement. Further, in FIGS. 15A and 15B, for a clearer view, members other than the intermediate tube member are appropriately omitted.

As shown in FIGS. 14 to 17, the overcoat tube 92 of the present embodiment includes an intermediate coupler 53 and a driving part 51B in place of the intermediate coupler 50 and the driving part 51 of the overcoat tube 90 of the second embodiment. Like the overcoat tube 90 of the second embodiment, the overcoat tube 92 is configured so that a distal end-side tube member 4 can be tilted in two axial directions centered on a pivotal center O on a proximal end-side tube member central axis A₂ of a proximal end-side tube member 2 with respect to the proximal end-side tube member central axis A₂. The following description will focus on differences between the second embodiment.

The intermediate coupler 53 includes a tubular part 46a, a flexible pipe 70, a pivotal support 73, a first holding member 71, a second holding member 72, and rotation transfer shafts 74 and 75 (a drive mechanism and a driving force transfer unit).

The tubular part 46a is configured similar to that of the third embodiment, and constitutes a driven part of the intermediate coupler 53.

As shown in FIGS. 15A and 15B, a distal end side of the tubular part 46a is connected to a proximal end face 4d of the distal end-side tube member 4, as in the third embodiment. Further, the flexible pipe 70 is connected to a proximal end side of the tubular part 46a.

The flexible pipe 70 is a flexible intermediate tube member for communicating insertion passages 4a and 2a of the distal and proximal end-side tube members 4 and 2. The flexible pipe 70 is configured so that a distal end side thereof is connected to the proximal end side of the tubular part 46a, and so that a proximal end thereof is connected to a stepped part 2c. In the flexible pipe 70, an insertion passage 70a having a size that is equivalent to that of the through-hole 46d is provided. Thereby, the insertion passage 70a communicates the insertion passages 4a and 4a with each other.

As a configuration of the flexible pipe 70, an arbitrary soft tube having such flexibility that the insertion passage 70a does not collapse even when curved or bent, such as a soft tube formed of a rubber or a synthetic resin, may be employed. In the present embodiment, a synthetic resin tube formed in a bellows shape is employed.

A connecting position of the flexible pipe 70 becomes approximately the same axis as the distal end-side tube member central axis A₄ at the distal end side, and the proximal end-side tube member central axis A₂ at the proximal end side.

However, the flexible pipe 70 has flexibility without a certain curving center or bending center. For this reason, by merely being connected by the flexible pipe 70, a positional relationship between the distal end-side tube member central axis A₄ and the proximal end-side tube member central axis A₂ is not fixed.

The pivotal support 73 constitutes a support of the intermediate coupler 53. As such, as shown in FIG. 16, the pivotal support 73 is a tubular part whose external form has a tetragonal prism shape provided on the stepped part 2c in the same axis as the proximal end-side tube member central axis A₂. In the present embodiment, sides of the rectangular external form of the pivotal support 73 are disposed to be approximately parallel to four respective sides constituting an external form of the stepped part 2c.

The pivotal support 73 has an axial position within a range in which the flexible pipe 70 is approximately covered from the stepped part 2c.

The pivotal support 73 is provided with a pair of holes 73b in lateral surfaces thereof which are through-holes centered on the y axis (vertical axis of FIG. 16) that is one of two axes perpendicular to each lateral surface and the proximal end-side tube member central axis A₂ on a plane perpendicular to the proximal end-side tube member central axis A₂. Further, the pivotal support 73 is provided with a pair of holes 73c that are through-holes centered on the x axis (horizontal axis of FIG. 16) that is the other of the two axes.

As shown in FIGS. 14 and 16, the first holding member 71 is configured of a frame member that includes U-shaped arm parts 71c and 71d that are disposed apart in the x-axial direction in a U shape when viewed from the side (x-axial direction) and that are provided in a plane-symmetrical shape with respect to a central plane of the y-axial direction, tabular lateral plate parts 71a and 71b that connect U-shaped openings to each other at proximal end sides of the U-shaped arm parts 71c and 71d in the x-axial direction, and a pair of pivot shafts 71g that are erected toward the negative-directional side of the y axis (lower side shown in FIG. 16) and the positive-directional side of the y axis (upper side shown in FIG. 16) at intermediate portions of a lengthwise direction (x-axial direction) of the lateral plate parts 71a and 71b and that have the same axis as the y axis.

Here, the lateral plate parts 71a and 71b have a positional relationship in which the lateral plate part 71a is disposed at the positive-directional side of the y axis.

In U-shaped curved portions of the U-shaped arm parts 71c and 71d, portions opposite each other are provided with holding surface parts 71e and 71f that are made up of planes that are parallel to each other. An x-axial distance between the holding surface parts 71e and 71f is approximately equal to an x-axial width Wₓ of the tubular part 46a, and the tubular part 46a can be slidably sandwiched between the holding surface parts 71e and 71f at positions spaced apart from the central axis of the pivot shafts 71g.

The holding surface parts 71e and 71f have such a size as to come into surface contact with the tubular part 46a at any position, and allow the tubular part 46a to be sandwiched therebetween such that the distal end-side tube member central axis A₄ is kept parallel to the holding surface parts 71e and 71f.

Each pivot shaft 71g is pivotably fitted into each hole 73b from an outer circumferential side of the pivotal support 73. For this reason, the first holding member 71 is coupled to be able to pivot around the y axis with respect to the pivotal support 73 by the pivot shafts 71g and the holes 73b.

Further, the lateral plate part 71a is provided with a rotation transfer groove part 71h in a lateral surface thereof at the positive-direction side of the y axis. The rotation transfer groove part 71h is engaged with a coupler 74a of the rotation transfer shaft 74 to be described below, and has the same axis as the pivot shafts 71g.

A shape of the rotation transfer groove part 71h when viewed from the top may be an arbitrary shape corresponding to a shape of the coupler 74a. However, in the present embodiment, the shape of the rotation transfer groove part 71h is a regular hexagonal shape by way of example.

The second holding member 72 is configured of a frame member that includes U-shaped arm parts 72c and 72d that are disposed apart in the y-axial direction in a U shape when viewed from the side (y-axial direction) and that are provided in a plane-symmetrical shape with respect to a central plane of the y-axial direction, tabular lateral plate parts 72a and 72b that connect proximal ends of the U-shaped arm parts 72c and 72d to each other in the y-axial direction, and a pair of pivot shafts 72g that are erected toward the negative-directional side of the x axis (left side shown in FIG. 16) and the positive-directional side of the x axis (right side shown in FIG. 16) at middle portions of a lengthwise direction (y-axial direction) of the lateral plate parts 72a and 72b and that have the same axis as the x axis.

Here, the lateral plate parts 72a and 72b have a positional relationship in which the lateral plate part 72a is disposed at the positive-directional side of the x axis.

The second holding member 72 has such external dimensions that the U-shaped arm parts 71c and 71d of the first holding member 71 are housed in the U-shaped inner sides of the U-shaped arm parts 72c and 72d, and furthermore that the U-shaped arm parts 71c and 71d do not interfere with the inner sides of the U-shaped arm parts 72c and 72d even when the first holding member 71 pivots around the y axis.

Thereby, a movable region of the first holding member 71 is disposed within that of the second holding member 72. Here, the movable region refers to an entire space region in which the first and second holding members 71 and 72 sweep when pivoting.

In U-shaped curved portions of the U-shaped arm parts 72c and 72d, portions opposite each other are provided with holding surface parts 72e and 72f that are configured of planes that are parallel to each other. A y-axial distance between the holding surface parts 72e and 72f is approximately equal to a y-axial width W_{y} of the tubular part 46a, and the tubular part 46a can be slidably sandwiched between the holding surface parts 72e and 72f at positions spaced apart from the central axis of the pivot shafts 72g.

The holding surface parts 72e and 72f have such a width as to come into surface contact with the tubular part 46a, and allow the tubular part 46a to be sandwiched therebetween such that the distal end-side tube member central axis A₄ is kept parallel to the holding surface parts 72e and 72f.

Each pivot shaft 72g is pivotably fitted into one of the holes 73c from the outer circumferential side of the pivotal support 73. For this reason, the first holding member 71 is coupled to be able to pivot around the x axis with respect to the pivotal support 73 by the pivot shafts 72g and the holes 73c.

With this configuration, the first and second holding members 71 and 72 are coupled to be able to pivot around the x and y axes with respect to the pivotal support 73, respectively. For this reason, the first and second holding members 71 and 72 are allowed to pivot in the two axial directions passing through the pivotal center O that is a point of intersection of the x and y axes.

Further, the lateral plate part 72a is provided with a rotation transfer groove part 72h in a lateral surface thereof at the positive-direction side of the x axis. The rotation transfer groove part 72h is engaged with a coupler 75a of the rotation transfer shaft 75 to be described below, and has the same axis as the pivot shafts 72g.

A shape of the rotation transfer groove part 72h when viewed from the top may be an arbitrary shape corresponding to a shape of the coupler 75a. However, in the present embodiment, the shape of the rotation transfer groove part 72h is a regular hexagonal shape by way of example.

The rotation transfer shaft 74 (75) drives the first holding member 71 (second holding member 72) to pivot around the pivot shaft 71g (72g), and thus is a drive mechanism combined with a driving force transfer unit.

A configurations of the rotation transfer shaft 74 (75) is not particularly limited if it is a shaft member that has appropriate flexibility and can transfer rotation. However, in the present embodiment, a flexible shaft made by repetitively coiling an arbitrary plurality of steel wires is employed.

As shown in FIG. 16, an end of the rotation transfer shaft 74 (75) is provided with a coupler 74a (75a) for transferring a torque.

Further, the rotation transfer shaft 74 (75) is covered with a covering tube 76 on an outer circumference side thereof in order to prevent contact with another member when rotated.

The coupler 74a (75a) of one end side of the rotation transfer shaft 74 (75) is coupled to the rotation transfer groove part 71h (72h) of the lateral plate part 71a (72a), and is rotatably held via a bearing 78 provided on a tabular drive mechanism holding plate 77 (78) erected on the stepped part 2c along the lateral plate part 71a (72a).

Further, one end side of the covering tube 76 is fixed to the drive mechanism holding plate 77 (78).

As shown in FIG. 17, proximal end sides of the rotation transfer shaft 74 (75) and the covering tube 76 are coupled to the driving part 51B.

As shown in FIG. 17, the driving part 51B is configured such that the tubular member fixing plate 26b and the driving pulleys 28 and 30 are removed from a configuration of the driving part 51 of the second embodiment, and the driving part 51B includes a pair of speed reducers 80 that reduce speeds of rotation of motors 27 and 29, and a pair of fixing casings 81 that couple the other end sides of the rotation transfer shafts 74 and 75 to output sides of the speed reducers 80.

As configurations of the speed reducers 80, for example, speed reducers based on gear trains installed between rotary shafts 27a and 29a of the motors 27 and 29 and the couplers 74a and 75a of the rotation transfer shafts 74 and 75 may be employed.

Next, an operation of the overcoat tube 92 of the present embodiment will be described focusing on an operation of the intermediate coupler 53.

According to the intermediate coupler 53, the first holding member 71 is pivotably coupled with respect to the pivotal support 73 and the proximal end-side tube member 2, to which the pivotal support 73 are provided, by the pivot shafts 71g that are provided on the same axis as the y axis that is the first pivotal axis perpendicular to the proximal end-side tube member central axis A₂. Further, the second holding member 72 is coupled to be able to pivot around the x axis that is the second pivotal axis orthogonally intersecting with the proximal end-side tube member central axis A₂ and the y axis at the pivotal center O.

Further, the holding surface parts 71e and 71f of the first holding member 71 (holding surface parts 72e and 72f of the second holding member 72) constitute a slit in which the tubular part 46a that is the lateral portion of the driven part is sandwiched in a circumferential direction of a pivotal circle of the first holding member 71 (second holding member 72) at positions spaced apart from the first pivotal axis (second pivotal axis).

In this case, the holding surface parts 71e and 71f (72e and 72f) come into surface contact with the tubular part 46a. Thereby, even during pivoting, the positional relationship parallel to the distal end-side tube member central axis A₄ is maintained, and thus the tubular part 46a is tilted in the two axial directions centered on the rotational axis O. Here, since the tubular part 46a and the proximal end-side tube member 2 are coupled via the flexible pipe 70, the flexible pipe 70 is curved inside the pivotal support 73 after the pattern of the tilted state of the tubular part 46a, and the tilting motion can be performed in a state in which the insertion passages 4a and 2a of the distal and proximal end-side tube members 4 and 2 are communicated by the insertion passage 70a.

Accordingly, the present embodiment is an example in which, even when the distal end-side tube member 4 and the proximal end-side tube member 2 are not coupled by the joint structure having the pivotal center O, the distal end-side tube member 4 is configured to be able to be tilted in the two axial directions centered on the pivotal center O with respect to the proximal end-side tube member 2 by the functions of the first and second holding members 71 and 72.

According to the intermediate coupler 53, without providing the joint structure having a complicated configuration in order to tilt around one point, the overcoat tube 92 can be configured to be able to be tilted around one point in the two axial directions by the flexible pipe 70 having a simple configuration.

In the second to fourth embodiments, the case in which two drive systems and a dual transfer system are provided so that the drive mechanism and the driving force transfer unit perform the tilting in the two axial directions has been described by way of example. However, the drive mechanism and the driving force transfer unit for tilting in only one axial direction may be provided, and the other axial direction may be set as a free tilting or fixed tilting angle.

Further, in the modified example of the second embodiment, the configuration in which the pinion gears and the racks are used as the driving part moving the rod-like members forward or backward has been described by way of example. However, as long as the rod-like members can move forward or backward, the driving part is not limited to this configuration. For example, a screw feed mechanism may be employed.

Further, in the second to fourth embodiments, the case in which the driving force supply unit supplying the driving force to the driving force transfer unit is installed outside the proximal end-side tube member has been described by way of example. However, the driving force supply unit may be mounted in the intermediate coupler. For example, a small motor may be mounted in the intermediate coupler.

Further, the case in which the driving force supply unit employs the motor has been described by way of example. However, the driving force may be configured to be supplied manually. For example, the driving pulleys 28 and 30 of the driving part 51 may each be provided with a handle to be able to be rotated manually. Further, the driving members 41 of the driving part 51A may be configured to manually move forward or backward.

Further, in the modified example of the third embodiment, the case of the drive mechanism formed by a combination of the lateral-portion pressing part 66 and the elastic member 67 has been described by way of example. However, another lateral-portion pressing part 66 may be provided in place of the elastic member 67, and the tubular part 46a may be configured to be sandwiched by the pair of lateral-portion pressing parts 66. The lateral-portion pressing parts 66 may move forward or backward in the opposite directions such that an opposite interval between the lateral-portion pressing parts 66 is kept constant. Thereby, the tubular part 46a may be configured to be tilted.

Further, in the above description, when the joint structure is provided, the configuration in which, like the ball joint, the communicating through-hole is provided therein, and thereby the distal end-side tube member and the proximal end-side tube member are communicated each other, and the configuration in which, like the universal joint or the gimbal joint, the plurality of through-holes are located in the intermediate coupler adjacent to one another without being continued have been taken by way of example. In this way, the intermediate coupler is sufficient if it can connect the insertion passages of the distal and proximal end-side tube members in the sense that the orifice thereof into which the medical instrument can be inserted is secured in the axial direction.

However, even when the universal joint or the gimbal joint is used, the intermediate tube member such as the flexible pipe 70 of the fourth embodiment is provided, and thereby the continuous tubular insertion passage may be configured to be formed inside the intermediate coupler.

Further, all the components described in each embodiment and each modified example may be carried out by appropriate combination or removal within the technical spirit of the prevent invention.

For example, the joint structure described in the first embodiment may properly employ the joint structure of the overcoat tube of the second or third embodiment.

Further, in the case in which the joint structure described in the fourth embodiment is not included, the drive mechanism and the driving force transfer unit may be removed and carried out as in the first embodiment.

In addition, each rotation transfer shaft that is the drive mechanism and driving force transfer unit of the fourth embodiment may be properly applied as the drive mechanism and the driving force transfer unit of the second or third embodiment.

Further, in the first embodiment, the case in which the support and the driven part of the joint structure are integrally provided on the proximal end-side tube member and the distal end-side tube member respectively has been described by way of example. However, like the inner case 19 of the second embodiment, to configure and couple a separate member for the distal end-side tube member 4, the support may be configured as a separate member for the proximal end-side tube member, the driven part may be configured as a separate member for the distal end-side tube member, and the support and the driven part may be configured to be coupled and fixed.

Further, the aforementioned ball joint is a joint structure, and includes:

a male joint part that has a convex spherical engaging surface and is provided on an outer circumference side of an end of one of a support and a driven part; and

a female joint part that has a concave spherical engaging surface slidably engaged with the convex spherical engaging surface of the male joint part and is provided on an outer circumference side of an end of the other of the support and the driven part,

wherein the male joint part and the female joint part have through-holes formed therein and communicating an insertion passage of the support and an insertion passage of the driven part with each other.

Further, the aforementioned universal joint is a joint structure, and includes:

a case member that has a through-hole formed in a central portion thereof and is disposed at an inner side of a support and an inner side of a driven part;

a first pivotal part that couples an outer circumference of the case member and the driven part to be able to pivot around a first pivotal axis that passes through the center of the case member and is perpendicular to a central axis of the through-hole of the case member; and

a second pivotal part that couples the outer circumference of the case member and the support to be able to pivot around a second pivotal axis that passes through the center of the case member and is perpendicular to a central axis of the through-hole of the case member and the first pivotal axis,

wherein the insertion passages of the distal and proximal end-side tube members are connected via the through-hole.

Further, the aforementioned gimbal joint is a joint structure, in which

a support and a driven part have a case shape provided on the same axis as the insertion passages, and one of the support and the driven part becomes an outer case that is allowed to be disposed outside the other of the support and the driven part,

the other of the support and the driven part becomes an inner case that is allowed to be disposed outside the one of the support and the driven part,

the joint structure includes:

an intermediate case member that is disposed at an inner side of the outer case and an outer side of the inner case;

an outside pivotal part that couples the intermediate case member and the outer case to be able to pivot around a first pivotal axis that is provided on a plane perpendicular to a central axis of the intermediate case member; and

an inside pivotal part that couples the intermediate case member and the outer case to be able to pivot around a second pivotal axis that is provided on the plane perpendicular to the central axis of the intermediate case member to be perpendicular to the first pivotal axis.

While preferred embodiments of the invention have been described and illustrated above, it should be understood that these are exemplary of the invention and are not to be considered as limiting. Additions, omissions, substitutions, and other modifications can be made without departing from the scope of the present invention. Accordingly, the invention is not to be considered as being limited by the foregoing description, and is only limited by the scope of the appended claims.

What is claimed is:

1. An overcoat tube configured to insert a distal end side thereof into a body cavity to secure an insertion passage through which a medical instrument is introduced from a proximal end side thereof into the body cavity, the overcoat tube comprising:
a distal end-side tube member forming the insertion passage at the distal end side;
a proximal end-side tube member forming the insertion passage at the proximal end side; and
an intermediate coupler that couples the distal end-side tube member to the proximal end-side tube member so that the distal end-side tube member is capable of being tilted in two axial directions centered on one point with respect to the proximal end-side tube member and connects the insertion passages of the distal and proximal end-side tube members.

2. The overcoat tube according to claim 1, wherein the intermediate coupler includes:
a support provided at a distal end side of the proximal end-side tube member;
a driven part provided at a proximal end side of the distal end-side tube member; and
a joint structure that couples the driven part to the support so that the driven part is capable of being tilted in two axial directions centered on one point with respect to the support.

3. The overcoat tube according to claim 2, wherein the joint structure includes a ball joint in which a through-hole is formed.

4. The overcoat tube according to claim 2, wherein the joint structure includes a universal joint in which a through-hole is formed.

5. The overcoat tube according to claim 2, wherein the joint structure includes a gimbal joint in which a through-hole is formed.

6. The overcoat tube according to claim 2, wherein:
the intermediate coupler includes a drive mechanism that drives a driving target configured of a movable member or the driven part in the joint structure to thereby tilt the driven part with respect to the support; and
the drive mechanism is connected with a driving force transfer unit that is remotely manipulated from an outside of the proximal end-side tube member to transfer a driving force.

7. The overcoat tube according to claim 6, wherein:
the drive mechanism includes a pulley that is fixed to the driving target and is pivotably supported on an axis perpendicular to a tilting central axis; and
the driving force transfer unit includes a wire wound on the pulley.

8. The overcoat tube according to claim 6, wherein:
the drive mechanism includes a pinion gear that is fixed to the driving target and is pivotably supported on a tilting central axis; and
the driving force transfer unit includes a rack engaged with the pinion gear, and a rod-like member moving the rack forward or backward in a given direction.

9. The overcoat tube according to claim 6, wherein:
the drive mechanism includes a link mechanism that is fixed to the driving target at one end thereof and is coupled to the driving force transfer unit at the other end thereof; and
the driving force transfer unit includes a rod-like member that moves a link member forward or backward at the other end side of the link mechanism in a given direction.

10. The overcoat tube according to claim 2, wherein:
the intermediate coupler includes: a movement restriction member that is provided on the proximal end-side tube member and restricts positions of lateral portions of the driven part at positions spaced apart from a tilting center of the joint structure; and a drive mechanism that drives the movement restriction member to thereby tilt the driven part with respect to the support; and
the drive mechanism is connected with a driving force transfer unit that is remotely manipulated from an outside of the proximal end-side tube member to transfer a driving force.

11. The overcoat tube according to claim 10, wherein the movement restriction member is coupled to be able to pivot with respect to the proximal end-side tube member, and includes a slit in which the lateral portions of the driven part are sandwiched in a circumferential direction of a tilting circle.

12. The overcoat tube according to claim 10, wherein
the movement restriction member includes a lateral-portion pressing part that comes into contact with a lateral surface of the driven part and is provided to be able to move forward or backward in an axial direction of the proximal end-side tube member; and a movement guide part that changes a position in a direction perpendicular to the axial direction of the lateral-portion pressing part based on a position of the axial direction of the lateral-portion pressing part, and
the driving force transfer unit includes a rod-like member that moves the lateral-portion pressing part forward or backward in the axial direction.

13. The overcoat tube according to claim 6, wherein
the drive mechanism includes two drive systems that tilt the driven part with respect to the support individually in the two axial directions, and
the driving force transfer unit includes a dual transfer system that transfers the driving force independently in the two axial directions.

14. The overcoat tube according to claim 6, further comprising a driving force supply unit that supplies the driving force to the driving force transfer unit and that is installed outside of the proximal end-side tube member.

15. The overcoat tube according to claim 1, wherein the intermediate coupler includes:
a flexible intermediate tube member that spatially connects the insertion passages of the distal and proximal end-side tube members with each other;
a first holding member that is coupled to the proximal end-side tube member so as to be capable of pivoting around a first pivotal axis perpendicular to a central axis of the proximal end-side tube member with respect to the proximal end-side tube member, and that holds lateral portions of a proximal end of the distal end-side tube member to be capable of being tilted around the first pivotal axis; and
a second holding member that is coupled to the proximal end-side tube member so as to be capable of pivoting around a second pivotal axis perpendicular to the central axis of the proximal end-side tube member and the first pivotal axis at one point with respect to the proximal end-side tube member, and that holds the lateral portions of the proximal end of the distal end-side tube member to be capable of being tilted around the second pivotal axis.

16. The overcoat tube according to claim 15, wherein:
the first and second holding members each include a drive mechanism that is driven to be capable of pivoting with respect to the proximal end-side tube member, and
the drive mechanism is connected with a driving force transfer unit that is remotely manipulated from an outside of the proximal end-side tube member to transfer a driving force.
